(19) United States
(12) Patent Application Publication
(10) Pub. No.: US 2017/0329929 A1
(43) Pub. Date: Nov. 16, 2017

(54) VIRTUAL PATHOLOGY FOR DERMATOLOGY

(71) Applicant: Sensus Healthcare LLC, Boca Raton, FL (US)

(72) Inventor: Kalman Fishman, Boca Raton, FL (US)

(21) Appl. No.: 15/592,958

(22) Filed: May 11, 2017

Related U.S. Application Data
(60) Provisional application No. 62/334,897, filed on May 11, 2016.

(51) Int. Cl. G06F 19/00; A61B 5/00; A61N 5/10

(52) U.S. Cl. CPC G06F 19/3437; G06F 19/3487; G06F 19/3481; G06F 19/3456; G06F 19/3418; A61B 5/0077; A61N 5/103; A61B 5/444; A61B 5/0082; G06F 19/321

(57) ABSTRACT

Virtual Dermatopathology Regional Hub (VDRH) receives one or more of the Detailed Virtual Tissue Models generated at one or more of the Virtual Dermatology Workstations and selectively communicates each to at least one pathology site. Each pathology site includes one or more Dermatopathology Information Processing Workstations (DIPWs). Thereafter, the VPM receives from the one or more of the pathology sites a plurality of pathology reports generated using said DIPWs and respectively corresponding to the plurality of DVTMs. The VPM then selectively communicates the pathology reports to the imaging facility which generated respectively the DVTM upon which each said pathology report is based.

### CROSS-REFERENCE TO RELATED APPLICATION

The present application claims benefit of U.S. Patent Application Ser. No. 62/334,897 filed on May 11, 2016, which application is incorporated herein by reference in its entirety.

### FIELD OF THE INVENTION

The disclosure relates to systems, devices and methods for detecting and treating skin conditions such as skin cancers; more particularly it relates to detection and treatment of skin cancers by leveraging a virtual pathology network.

### BACKGROUND

Skin cancer is a type of cancer in which an abnormal growth of cells appears in the skin, and is the most commonly diagnosed type of cancer. The three most common malignant skin cancers are basal cell cancer, squamous cell cancer, and melanoma, each of which is named after the type of skin cell from which it arises. The chances of surviving skin cancer increase if it is detected early and treated appropriately.

For the most part skin cancer is viewed as a simpler problem than other types of cancer. Since it often appears directly on the surface of the skin it is thought of as more of a two-dimensional problem as opposed to a three-dimensional problem as in other types of cancer. So unlike other types of cancers, skin cancers are frequently treated without the use of advanced imaging equipment.

Practitioners frequently begin evaluating skin cancers by arranging for a patient to visit a treatment center and directly observing the lesion on the surface of the skin. The practitioner can make an initial evaluation of what type of cancer might be present based on the appearance of the lesion. Based on this initial evaluation, the practitioner may then perform a biopsy of the lesion to obtain a tissue sample. A pathologist specializing in the evaluation of skin cancer tissue types is usually located at some facility that is physically remote from the treatment center where the biopsy is performed. The treatment center may be tens or hundreds of miles from the location where the biopsy was performed. So the tissue sample obtained from the biopsy must be packaged and physically transported from the treatment center to the pathologist.

Once received by the pathologist, the package and sample must be carefully tracked to ensure that there is no confusion as to the particular patient from whom the biopsy sample originated. The pathologist, who specializes in analysis of skin lesions so as to determine the nature of the lesion, will study the sample to determine if it comprises a particular type of cancer. For example, the pathologist may determine that the tissue comprises a basal cell cancer, squamous cell cancer, or a melanoma. The pathologist must then prepare a report, making sure he properly identifies the sample which is the subject of such report. The report is then communicated back to the treatment center.

When the results of the biopsy are obtained at some later date, the practitioner may estimate a margin and depth of skin that must be excised to remove the lesion by surgical or other means. From the foregoing it will be appreciated that the process of diagnosing a potential skin cancer is a time consuming and labor intensive process. Accordingly, significant costs and delays are introduced to the skin cancer treatment process.

### SUMMARY OF THE INVENTION

A virtual dermatology system includes a Virtual Dermatopathology Regional Hub (VDRH) and a plurality of Virtual Dermatology Workstations (VDWs). The VDWs are each comprised of a hybrid imaging system which includes a structural imaging component adapted to image structural features of the skin lesion, an optical imaging component adapted to image functional features of the skin lesion, and an image fusion component. The image fusion component is adapted to combine the images obtained by the structural imaging component and the optical imaging component to form a 3D virtual model comprising the DVTM.

The VDRH is comprised of at least one processing element which implements a Virtual Pathology Manager (VPM). The VDRH is configured to communicate over at least one data network to receive at the VPM a plurality of Detailed Virtual Tissue Models (DVTMs) of imaged skin lesions from one or more VDWs. The VDRH selectively communicates from the VPM each of the plurality of DVTMs to one or more pathology sites, each comprising one or more Dermatopathology Information Processing Workstations (DIPWs) which are configured to display the DVTM for analysis by a pathologist. It then receives at the VPM from one or more of the pathology sites a plurality of pathology reports generated using said DIPWs and respectively corresponding to the plurality of DVTMs. Finally, the VPM communicates the received pathology reports to the imaging facilities which generated respectively the DVTM upon which each said pathology report is based.

An embodiment disclosed herein also concerns a method for virtual dermatology. A data network is used to receive at a Virtual Dermatopathology Regional Hub (VDRH) one or more of the DVTMs generated at one or more of the VDWs. A VPM receives the one or more DVTMs and selectively communicates each to at least one pathology site. Each pathology site includes one or more DIPW configured to display the DVTM for analysis by a pathologist. Thereafter, the VPM receives from the one or more of the pathology sites a plurality of pathology reports generated using said DIPWs and respectively corresponding to the plurality of DVTM. The VPM then selectively communicates the pathology reports to the imaging facilities which generated respectively the DVTM upon which each said pathology report is based.

### BRIEF DESCRIPTION OF THE DRAWINGS

FIG. 1A is a schematic of a high level overview of a virtual dermatology system.

FIG. 1B is schematic representations which shows how a Virtual Dermatopathology Regional Hub (VDRH) interacts with one or more virtual dermatology workstations (VDWs).

FIG. 1C is a schematic representation which shows how a VDRH interacts with one or more Dermatopathology Information Processing Workstations (DIPWs).

FIG. 1D is a schematic representation which shows how an exemplary VDRH interacts with one or more Radiation Therapy Planning (RTP) workstations.

FIG. 1E is a more detailed view of an exemplary virtual dermatology workstation which can reside in a treatment facility associated with a dermatology practice.

FIG. 2 is an exemplary embodiment of an image-guided radiotherapy system.

FIG. 3 is a schematic drawing of another embodiment of an image-guided radiotherapy system.

FIG. 4A is a flow diagram of a tumor imaging and quantification process.

FIG. 4B illustrates an exemplary method for generating a 3D model of a tumor.

FIG. 5 is flow diagram of a method of diagnosis and treatment.

FIG. 6 is an exemplary embodiment of a beam sensing component.

FIG. 7 is a diagram that is useful for understanding a beam projection created by using mask or shield.

FIG. 8 is a flow diagram of a process of treating a patient.

FIG. 9 is a diagrammatic representation of a machine in the form of a computer system within which a set of instructions, when executed, may cause the machine to perform any one or more of the methodologies discussed herein.

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

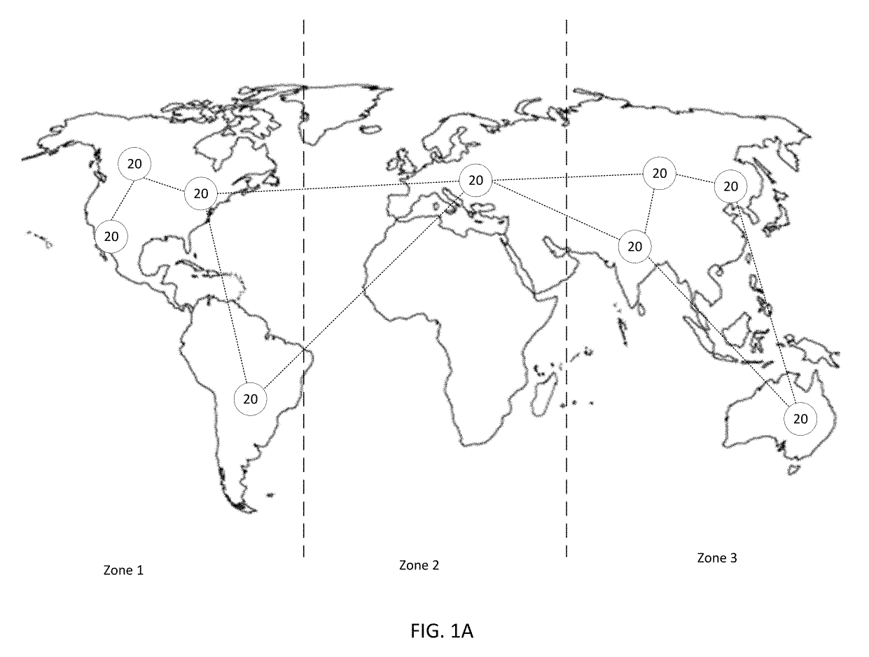

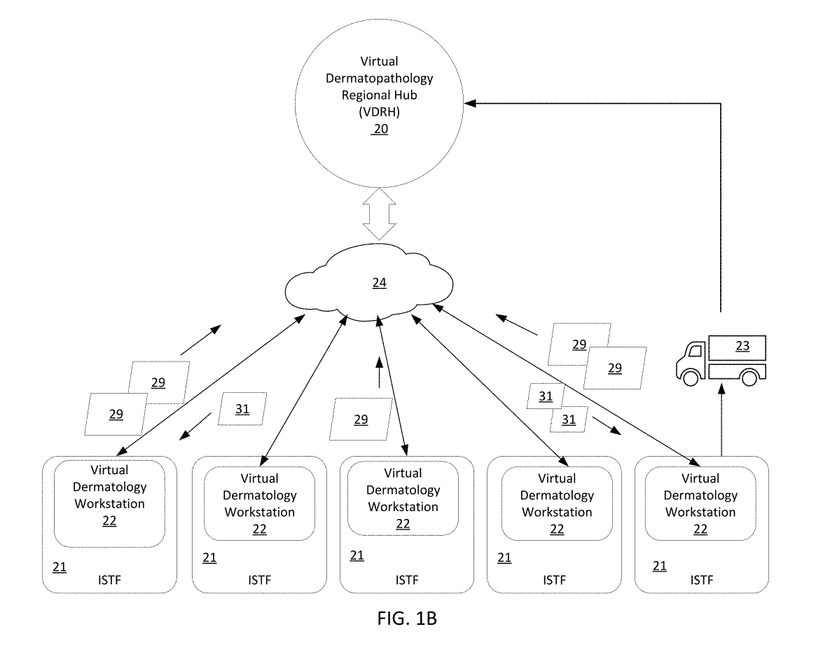

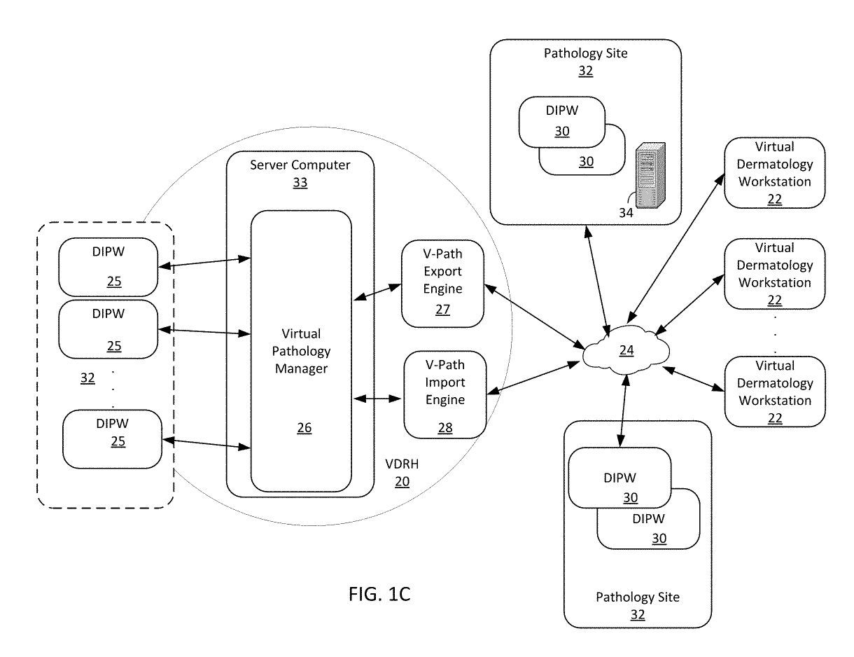

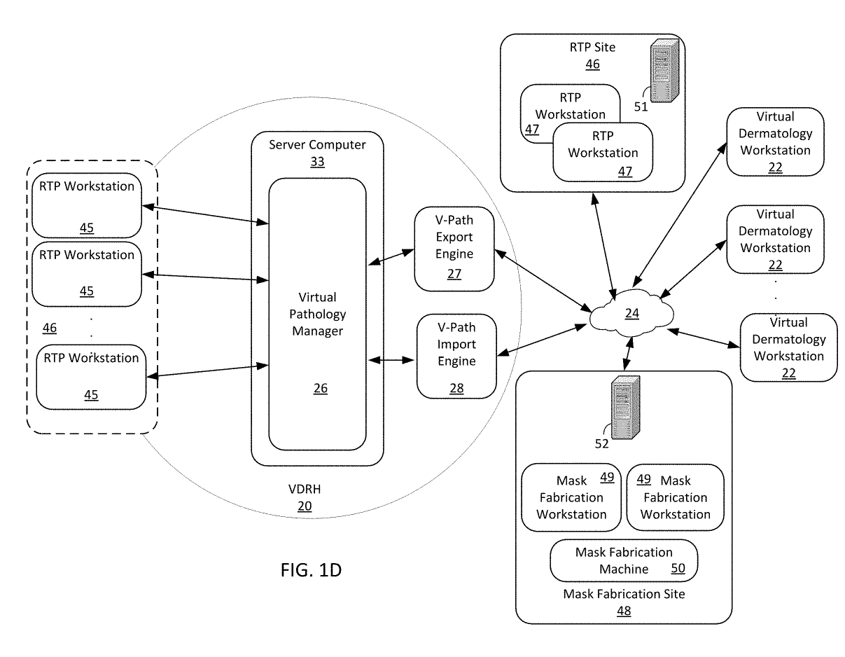

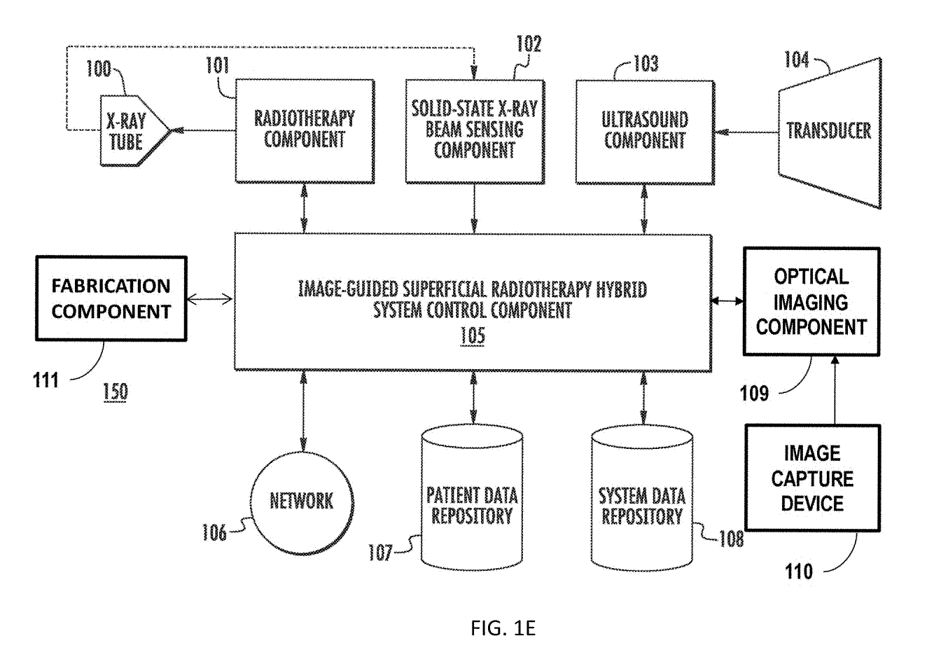

A virtual pathology network and method is disclosed to facilitate diagnosis and treatment of lesions in the field of cutaneous oncology. The network, which is best understood with reference to FIGS. 1A-1E, includes one or more Virtual Dermatopathology Regional Hubs (VDRHs) 20 which are geographically dispersed. For example, the VDRHs 20 can be spread as shown in FIG. 1A over a large geographic area which can include the entire world, or portions thereof. In some scenarios, each VDRH 20 can be comprised of a computer server. As shown in FIG. 1B the network also includes one or more imaging, sampling and treatment facilities (ISTFs) 21 which are geographically dispersed around each VDRH. Each ISTF will include at least one Virtual Dermatology Workstation (VDW) 22.

As shown in FIG. 1C, the network also includes one or more pathology sites 32, each comprising one or more Dermatopathology Information Processing Workstation (DIPW) 25, 30. As such, a pathology site 32 can in some scenarios include one or more central servers or network appliances 34 to facilitate communications between the VDRH and the DIPWs. A DIPW 25 can in some scenarios be co-located at the same facility with the VDRH 20. Alternatively, or in addition thereto, a DIPW 30 can be situated at a location which is geographically remote from the VDRH. Accordingly a pathology site 32 can in some scenarios be co-located with the VDRH 20 and in other scenarios can be geographically remote from the VDRH 20. The VDWs 22, the VDRHs 20 and the DIPWs 25, 30 are communicatively coupled by means of at least one data communication network 24, which in some scenarios can include a global communications network.

As shown in FIG. 1D, the network can also include one or more Radiation Therapy Planning sites 46, each comprising one or more RTP workstations 45, 47. As such, an RTP site 46 can in some scenarios include one or more central servers or network appliances 51 to facilitate communications between the VDRH and the RTP workstations. An RTP workstation 45 can in some scenarios be co-located at the same facility with the VDRH 20. Alternatively, or in addition thereto, an RTP workstation 47 can be situated at a location which is geographically remote from the VDRH. Accordingly an RTP site 46 as described herein can in some scenarios be co-located with the VDRH 20 and in other scenarios can be geographically remote from the VDRH 20. As shown in FIG. 1D, the VDWs 22, the VDRHs 20 and the RTP workstations 45, 47 are communicatively coupled by means of at least one data communication network 24, which in some scenarios can include a global communications network. Similarly, a mask fabrication site 48 can include one or more mask fabrication workstations 49, and a mask fabrication component 50. The mask fabrication site 48 can include one or more central servers or network appliances 52 to facilitate communications between the VDRH and the mask fabrication workstations.

The VDWs 22 are configured to capture data sufficient to create a detailed virtual tissue model (DVTM) 29 of a cutaneous lesion that is sufficient for diagnosis of a disease condition, and radiotherapy treatment planning (RTP). Once captured, the data comprising the DVTM 29 is encapsulated at the VDW 22 for transport in a data communication network 24. The encapsulated data can include information to facilitate tracking of the DVTM 29 through a virtual pathology network as described herein. For example a tracking number can be assigned to the DVTM. The encapsulated data can also include information concerning the origin of the detailed visual model. Such origin information can include the patient name, the anatomical location on the patient's body represented by the detailed visual model, the treating physician, the technician who captured the data, identification of the treatment facility where the detailed model was generated, and so on.

As shown in FIG. 1B, a DVTM 29 which is generated at a VDW 22 is communicated through the data communication network 24 to the VDRH 20. The VDRH mediates between one or more of the VDWs 22 and one or more of the DIPWs 25, 30 at the pathology sites. More particularly, a VDRH will receive data comprising the DVTM 29 and communicate same to one or more DIPWs 25, 30. A pathologist located at the pathology site can use DIPW 25, 30 to display the DVTM 29 for study and for generating a pathology report based on the DVTM. Data comprising a pathology report 31 is then communicated back through the VDRH 20 which routes it to the VDW 22 which originally generated the DVTM.

Each VDRH can include a Virtual Pathology Manager 26, a V-Path export engine 27, and a V-Path import engine 28. The Virtual Pathology Manager is configured to identify one or more of the pathology sites that is best suited to perform the analysis of each received DVTM 29. In some scenarios, the Virtual Pathology Manager 26 can be implemented in software which executes on a server computer 33. The V-path export engine and V-path import engine facilitate data encapsulation and/or de-encapsulation with respect to DVTMs 29 and pathology reports 31. These components can also facilitate encryption and decryption operations to ensure data integrity. In some scenarios functions associated with the Virtual Pathology Manager 26 can be integrated with functions performed by the V-Path export engine 27 and V-Path import engine 28. In some scenarios, modules similar to the V-Path export engine 27 and V-Path import engine 28 can be included in servers 34, 51, and 52.

The Virtual Pathology Manager 26 advantageously performs managing and scheduling operations so that the DVTMs 29 which are generated at the VDWs 22 are distributed among one or more pathology sites 32 where DIPWs 25, 30 are located. Consequently, timely and efficient pathology report generation is ensured. To facilitate the management and scheduling functions described herein, the Virtual Pathology Manager 26 will advantageously track certain information which is useful for managing the one or more pathology sites. For example, with respect to each pathology site the VDRH can track (1) the number of DVTMs which have been sent for study, (2) the date and time they were sent, (3) a backlog indicating the number of DVTMs sent to a particular pathology site for which pathology reports have not yet been generated, and (4) a number of pathologists and/or DIPWs available at each pathology site, and (5) the expertise of the pathologists who are present at a particular pathology site. Based on such information, the VDRH can make certain decisions about where a DVTM is to be sent for analysis when received from a VDW 22.

In some scenarios, the Virtual Pathology Manager 26 can perform a preliminary analysis of the DVTM 29 to automatically determine the most appropriate pathology site or DIPW 25, 30 for analyzing the hybrid image therein. For example, the most appropriate pathology site/DIPW 25, 30 can be determined in accordance with an automated preliminary analysis regarding the type of lesion. If a particular pathology site 32 is known to be have practitioners with certain expertise relating to the particular type of lesion, then the DVTM can be sent to that site on the basis of such known expertise.

According to a further aspect, the Virtual Pathology Manager 26 can determine the most appropriate pathology site on the basis of one or more pathology site load characteristics. For example, such load characteristics can include a current backlog of DVTMs to be analyzed by a particular site, the number of pathologists who are available to perform such image analysis, the number of DIPWs at the site, the accuracy of the site, the cost or fees charged by the pathology site for the analysis, the rate that the pathology sites is able to perform such analysis, and so on. Once the analysis has been completed at the pathology site, the results are communicated to the VDRH which forwards them to the VDW 22 from whence they originated.

In some scenarios, pathology sites 32 that are associated or known to a particular VDRH 20 may be backlogged with work and/or have insufficient resources to perform analysis of the DVTM. In some scenarios, the type of practitioner expertise that is needed to analyze a particular DVTM 29 may not be available in the pathology sites 32 that are known to or associated with a particular VDRH 20. In such scenarios, a first VDRH shown in FIG. 1A can forward the DVTM 29 to a second VDRH 20 at a different geographic location. The second VDRH can then determine if one of its associated pathology sites 32 has capacity and/or expertise to prepare a suitable pathology report. An arrangement as described herein can also facilitate more rapid turn-around times for completion of pathology reports, by taking advantage of time zone differences. Referring once again to FIG. 1A, an ISTF located in first geographic zone (e.g., Zone 1) can generate a DVTM late in the afternoon, at a time when few pathologists are available to analyze the DVTM for purposes of generating a pathology report. In such scenarios, the Virtual Pathology Manager 26 at a first VDRH 20 in a first zone (e.g., Zone 2) can forward the DVTM 29 to a second VDRH in a second zone (e.g., Zone 1) where the work day is just beginning. Accordingly, pathology results for DVTM can be easily obtained 24 hours a day.

In addition to the imaging methods employed to acquire the tumor topology and pathology data, and as an adjunct pathology data acquisition method, the integration of a non-invasive method to collect tissue samples can be incorporated and integrated in the process. In such scenarios, the non-invasive tissue sampling is advantageously performed at the ISTF 21 in the form of topical tissue sampling of the tumor on the surface of the skin. This method may be comprised of various tissue sampling means, such as micro-adhesives, scraping, or topical bio-sweeping of the sampled tumor tissue on the surface of the skin onto/into a sterile and protective medium in order to add another physiological dimension to the acquired metabolic and physiologic imaging data of the tumor.

Using one or more of the foregoing methods, physiological tumor tissue samples collected onto a specialized kit/container. These samples will be either analyzed and digitized on site, at the clinical practice, or packed and shipped to the VDRH. For example, FIG. 1B illustrates that these tissue samples can be packaged at an ISTF 21 and physically transported by vehicle 23 in a conventional manner to a pathology site 32 for analysis in conjunction with study of the DVTM. Accordingly, the process described herein can advantageously integrate actual physiological tissue sampling with the multi-modal imaging data which comprises the DVTM.

To facilitate generation of a DVTM 29, novel combinations of hybrid imaging are used. The hybrid imaging methods described herein are specifically advantageous to cutaneous oncology and will have various nuances specific to radiation therapy planning (“RTP”) oncology for skin cancer. Combined imaging methodologies provide a combination of anatomical and functional/metabolic data that facilitate the virtual pathology aspects of the various embodiments. The imaging methodologies allow a pathologist to gain a highly accurate understanding of the skin cancer problem at hand based exclusively on the acquired DVTM 29. This capability is facilitated at the ISTF 21 by using the VDW 22, which is sometimes referred to herein as a cutaneous oncology workstation. The VDW will advantageously use a unique combination of different imaging modalities to facilitate generation of the DVTM. In some scenarios, the DVTM 29 is comprised of a three-dimensional model of a lesion that is sufficient to enable a pathologist to identify cutaneous cancer tissue, and its type.

The hybrid imaging methodology advantageously includes a first imaging methodology that is optimal for imaging structural/anatomical features associated with a particular cutaneous lesion or malignancy, and a second imaging methodology that is optimal for obtaining functional or metabolic features associated with the cutaneous lesion. The imaging modalities are registered and fused to form a hybrid image or DVTM that combines the best features of both the first and second imaging methodology. The particular imaging technologies utilized are such that they can be practically and economically offered in a ISTF for skin cancer, such as a dermatologist office. Further, the imaging can be provided in a compact, maneuverable, and relatively low cost platform.

According to one aspect, the first imaging methodology which is used to facilitate virtual pathology as described herein is high frequency ultrasound (“HFU”). HFU is used to facilitate anatomical imaging of very fine and thin tissue as would be necessary for a pathologist to evaluate the structure of a potentially cancerous skin lesion. The HFU imaging modality captures structural information a remote pathologist would need to understand the structure of imaged cells at different skin depths.

HFU involves a pulse-echo technique which facilitate imaging of tissues within the human body. Pressure waves comprising high frequency sound pulses are transmitted into cutaneous tissue, giving rise to echoes when the signals encounter interfaces or reflectors. These interfaces or reflector surfaces are defined by variations in the acoustic impedance as between different tissue types within the skin which result from variations in tissue density. Echo signals resulting from these interfaces are received and then amplified so that they can be visually represented on a display. For example, in some scenarios stronger reflections are presented in lighter shades and weaker reflections can be displayed in darker shades. Consequently, the HFU imaging modality can facilitate differentiation of human tissue or cells based on the density of such cells or skin tissue. Cells in skin tissue that are associated with a lesion will have a different density as compared to healthy cells. Accordingly, the location and boundaries of a lesion can be identified by the pathologist in an image using the HFU imaging modality.

In some scenarios, the HFU imaging involves the acquisition of imaging data in a series of two-dimensional (2-D) slices which extend from the outer surface of the skin tissue toward one or more subcutaneous skin layers. A plurality of slices obtained in this way can be combined to render a three-dimensional (3-D) model of the imaged tissue volume. The volume in the areas between the 2-D slices can be extrapolated from the data associated with adjacent slices.

Other scanning or imaging methods can also be used to facilitate understanding of the anatomy or structural characteristics of the cancer. These methods can be used in place of or in addition to the HFU imaging methods described herein. For example, such methods can include relatively simple laser scanning techniques such as LIDAR (Light Detection and Ranging). Alternatively, more complex scanning methods such as Optical Coherence Tomography (OCT) can be used to obtain detailed structural data associated with the cancer. Unlike LIDAR, it will be appreciated that OCT can penetrate beneath the epidermis. Of course, OCT has some limits with regard to how deep it can penetrate beneath the epidermis, but it can provide a very detailed view of the topology and the anatomy and in some scenarios could be used in place of HFU imaging as described herein.

While the HFU imaging and/or other modalities described herein can identify the presence of a lesion and its structural boundaries, this structural information would be insufficient by itself to allow a pathologist to adequately identify the biological nature of the lesion. For example, the HFU imaging modality can be inadequate to identify whether a particular lesion is a basal cell carcinoma (BCC), a squamous cell carcinoma (SCC) or a melanoma. The HFU can also be inadequate to allow a remote pathologist to differentiate between a skin cancer of some type and a mole or macule. In order to overcome this limitation, a second imaging methodology is used.

The second imaging methodology is an optical imaging method advantageously selected to facilitate the visualization of functional or biological data associated with the cells which are being imaged. According to one aspect, the optical imaging methodology can be chosen to facilitate the optical acquisition of a two-dimensional image at one or more different skin depths. Accordingly, functional/biological imaging data for a particular skin cancer can be acquired at a surface of the skin and/or at one or more different subcutaneous layers or depths beneath the surface of the skin.

According to one aspect, the optical imaging methodology is a spectroscopic imaging method. For example, the optical imaging methodology can comprise a multispectral imaging method that captures image data at a plurality of optical frequencies. Such multispectral imaging can include optical energy from the visible portion of the light spectrum but can also include optical energy from frequencies beyond the visible light range (e.g. infrared and near ultraviolet). Alternatively, the optical imaging methodology can comprise hyperspectral imaging wherein optical information is captured from across the electromagnetic spectrum at each pixel in the captured image. As a further alternative, the spectroscopic imaging method can comprise Raman spectroscopy which captures changes in the frequency of photons in monochromatic light which result from interaction with skin tissue.

The nature of a particular skin lesion will be consistent throughout the lesion, so it is not necessary to capture the functional data associated with the cells at all depths. Once the type of cancer has been determined using the second imaging methodology, all of the cells which are identified as being associated with the lesion can be marked accordingly in a visual model of the lesion. As noted above, the UHF imaging can differentiate such cells as having a density different from adjacent healthy/normal skin cells. Accordingly, in a subsequent data fusion process, the cells having a first density associated with a cancerous lesion can be highlighted, marked or otherwise displayed in a certain way to differentiate them from the adjacent healthy skin cells. For example, the cancerous skin cells can be displayed in a different color as compared to normal skin cells. In some scenarios, the particular color of the cancerous cells can correspond to the type of cancer which has been imaged. For example, BCC cells could be presented in yellow, SCC cells could appear blue, and melanoma cells could be presented in red.

In some scenarios, the second imaging methodology described herein can comprise biomarker optical imaging (BOI). Various types of BOI imaging methods can be used for this purpose. The biomarker can be administered to a patient by any suitable means. For example, the biomarker can be applied topically, injected hypodermically at the site of a suspected cancer, or administered intravenously. With BOI, a biomarker (typically a fluorescent biomarker) is used that emits photons when a certain chemical/biological response or compounds are present. For example, a biomarker could identify high metabolic rate or a specific receptor based on its fluorescent activities or release of photons. An optical imager can be used collect those photons and form an image. The biomarkers used for the purposes of the imaging described herein are advantageously selected so that a cancerous skin lesion will appear to light up or illuminate in captured images so that the cells can be more easily identified when imaged using the second imaging method as described herein.

BOI imaging as described herein can facilitate graphically representing an area in which malignant cells are present. BOI imaging methods and systems are known in the art and therefore will not be described here in detail. However, it will be appreciated that the BOI imaging can be obtained using any type of biomarker and any suitable type of imaging system to capture the image.

Referring now to FIG. 1E there is illustrated a high level view of an exemplary VDW system 150 and its main sub-modules for generating the DVTM 29. The exemplary VDW system 150 provides multiple imaging devices to generate the DVTM. The VDW system 150 can further include software to analyze and combine data and images produced by the imaging devices to form the DVTM 29. Although not required for implementation of a virtual dermatology pathology system as described herein, the VDW system 150 can also include a radiotherapy component.

As shown in FIG. 1E, the VDW system 150 can advantageously include a radiotherapy component 101 with X-ray tube 100, a solid-state X-ray beam sensing component 102, an ultrasound component 103 with a transducer 104, an optical imaging (OI) component 109 with an associated image capture device (ICD) 110. The system also includes a system control component 105 for guiding the radiotherapy of the radiotherapy component 101 based on images and data obtained from the ultrasound component 103, transducer 104, OI component 109, and ICD 110. The system control component 105 can also work with the solid-state X-ray beam sensing component 102 to ensure that the radiotherapy is of the appropriate intensity, depth and size. In some embodiments, the system can further include a template or shield fabrication component 111.

The ultrasound component 103 can include control circuitry, system drivers, operation control software, and a transducer 104, which can be a high frequency ultrasonic transducer, for superficial epidermis, dermis-level and/or subcutaneous tissue anatomical imaging. The ultrasound component 103 communicates with the software of the system control component 105 via a bus and system drivers. The ultrasound component 103 and transducer 104 are provided in exemplary system 150 to provide structural or anatomical data without exposing a subject to ionizing radiation. However, the present disclosure contemplates that ultrasound component 103 and transducer 104 can be replaced or supplemented in VDW system 150 with components for supporting any other types of imaging techniques that also do not utilize ionizing radiation. For example, optical coherence tomography or laser range scanning (LIDAR), to name a few.

The optical imaging component 109 can include control circuitry, system drivers, operation control software, and an image capture device 110, for superficial epidermis, dermis-level and/or subcutaneous tissue functional imaging. According to one aspect, the optical imaging component is a spectroscopic imaging device. For example, the optical imaging component can comprise a multispectral imaging device that captures image data at a plurality of optical frequencies. Such multispectral imaging component can be configured to utilize optical energy from the visible portion of the light spectrum for imaging purposes, but can also utilize optical energy from frequencies beyond the visible light range (e.g. infrared and near ultraviolet). Alternatively, the optical imaging component can comprise a hyperspectral imaging device wherein optical information is captured from across the electromagnetic spectrum at each pixel in the captured image. As a further alternative, the spectroscopic imaging device can be configured for Raman spectroscopy which captures changes in the frequency of photons in monochromatic light which result from interaction with skin tissue.

The optical imaging component 109 communicates with the software of the system control component 105 via a bus and system drivers. The present disclosure contemplates that optical imaging component 109 and the image capture device 110 can be replaced or supplemented in system 150 with components for supporting any other types of imaging techniques for extracting molecular or functional information from tissues. For example, biomarkers can be used to enhance the usefulness of the optical imaging methods described herein. As is known, a biomarker can involve a substance which is introduced to a tissue to facilitate the identification of a disease condition such as cancer. According to one aspect, a biomarker can include any substance introduced to a skin tissue which can be used to induce visually or optically detectable changes that can facilitate identification of cancerous cells. Any biomarker now known or known in the future can be used in conjunction with the optical imaging component 109 and image capture device 110 provided that it can help facilitate identification of functional data pertaining to skin tissue under observation.

Within the VDW system 150 the HFU imaging and the optical imaging is registered and then combined or superimposed to obtain a hybrid image. The system will use the images from the first and second image source to create the DVTM 29 which is comprised of the hybrid HFU/optical image that will represent a skin tissue sample. Cancerous cells having a different density from healthy cells can be rendered in a color corresponding to the particular type of cancer that has been detected. The image can represent the cancer in three dimensions, in a plurality of cross-sectional views and/or in a pseudo-three dimensional representation of a skin volume comprised of two dimensional image slices taken along two separate orthogonal axes. This DVTM, which is in effect a virtual tissue sample, can then be communicated to a VDRH 20. In some scenarios, the actual registration and superimposing operations can optionally be performed at the VDRH 20 and/or at a DIPW 25, 30, in which case the DVTM 29 is comprised of the multiple types of imaging data, and the necessary registration information.

The superficial radiotherapy component 101 can be utilized to treat any tumors, lesions or areas where analysis or diagnosis determines that treatment is needed. The superficial radiotherapy component 101 delivers collimated and focused x-ray photon particles to treatment areas. It is contemplated that the X-ray tube 100 will be selected so that is optimized for superficial cutaneous interaction with skin tissue, and has minimal effects at deeper tissue depths. For example a conventional superficial radiation therapy (SRT) type of X-ray unit can be used for this purpose. As will be appreciated, an SRT type of X-ray unit produces low energy X-rays that are suitable to treat skin conditions as hereinafter described.

The solid-state X-ray beam sensing component 102 can monitor the beam output of the radiotherapy component 101 and x-ray tube 100, along with overall system stability and yield. The solid-state X-ray beam sensing component 102 is mounted underneath the X-Ray tube 100 and is moved in front of the tube when the system 150 needs to be tested for quality control, or overall system 150 diagnosis purposes. Otherwise, it is retracted back in its home position, away from the X-ray tube 100 and the X-ray beam in order not to interfere during a normal operating mode. In addition to or as an alternative to using a X-ray based radiotherapy in VDW system 150, any other types of radiotherapy can be used. Thus, the components for radiotherapy can be selected to support photon-based radiotherapy (e.g., x-rays and gamma rays), particle-based radiotherapy (e.g., electrons, protons, neutrons, carbon ions, alpha particles, and beta particles), or any combinations thereof.

In an exemplary operation, the VDW system 150 utilizes the ultrasound component 103 with a transducer 104 to scan and image a tissue volume of interest, such as a volume of the skin with a lesion, to obtain structural or anatomical information about the region of interest. The system then utilizes the optical imaging component 109 with image capture device 110 to optically scan and image the same volume to obtain functional and/or metabolic information pertaining to the skin tissue or portions thereof. As used herein, the functional and/or metabolic information referenced herein can include any information pertaining to the biological function, behavior or processes at work in a particular cell or group of cells. The ultrasound and optical scanning processes will be described below in further detail. However, it should be understood that each scanning method will advantageously provide image data sufficient in combination to produce a DVTM 29 comprised of a three-dimensional representation of the scanned volume of tissue. In some scenarios, the three-dimensional representation produced by the ultrasound and optical image scanning methods described herein can each individually comprise a plurality of two-dimension image slices taken along one or more orthogonal axes, which can be combined to form a three-dimensional image.

A registration process is used to facilitate alignment of the image data acquired using the ultrasound and optical scanning methods. After the region of interest has been scanned and imaged by the system 150, the image data is processed by the VDW system's software. The image data acquired using the ultrasound and optical scanning methods can be registered and then fused or merged to form a single image comprising the DVTM. In the fused image, the image data acquired by using ultrasound is basically superimposed over the image data acquired by using the optical scanning method described herein. The resulting DVTM is a hybrid image which includes detailed anatomical and/or structural data for the skin cancer with the functional data for the same tissue volume superimposed.

The DVTM 29 can be used by a pathologist to analyze and quantify the tumor and subsequently prepare a treatment plan that is derived from the actual tumor parameters, such as volume, circumference, penetration depth, and tissue density. Once the tumor analysis and quantification are complete, the pathology report is delivered to the ISTF 21. The software in the VDW system 150 can provide analytical guidance to deliver the most accurate and appropriate superficial radiotherapy pertaining to the scanned and analyzed tumor. The therapy is then delivered by the integrated superficial radiotherapy component 101. The system's software documents the entire diagnosis and treatment cycle and archives the patient data on a patient data repository 107 and the overall system 150 functionality log on a system data repository 108.

The VDW system 150 is controlled and operated by the system control component 105, which can include a central computer with a motherboard that runs operation and control software with various parallel and connected boards that allow it to control, communicate, and monitor the various sub-components and modules of the system 150. This achieves harmonious functionality between the main clinical components of the system 150. These components can include the superficial radiotherapy component 101, which provides radiotherapy treatment, the ultrasound component 103, which is utilized to scan and acquire the anatomy and topology of a patient's skin area, and the optical imaging component 109 which facilitates functional analysis of the tissue. The system control component 105 can be connected with data repositories, including a patient data repository 107 and a system data repository 108. The system 150 can also be connected to a network 106, such as a local area network, a wide area network and/or the Internet, which allows for clinical and system data exchange with remote systems or networks.

In some scenarios, the system control component 105 can be configured to output a two dimensional pattern for a template or shield to be used during radiation treatment for masking or shielding certain portions of a patient's skin. The two-dimensional pattern can be output to a user in the form of an image or pattern that is suitable to facilitate manually marking and cutting a metal plate which can be used as a shield or template in accordance with a radiation therapy treatment. Alternatively, the control component 105 can output the shield pattern in a data file format which is suitable for controlling a fabrication machine. In some scenarios, a fabrication machine 111 can be included as part of the system 150. One example of a fabrication machine 105 that can be used for this purpose can include a tabletop computer numerically controlled (CNC) router (e.g., a CNC machine). However, the embodiments are not limited in this regard and the fabrication machine 111 can also comprise a 3D printer that is capable of 3D metal printing. Thereafter, the fabricated shield or template can be fabricated by the fabrication machine 111 so that it is available for use in treatment of a patient.

The patient data repository 107 and the system data repository 108 can be a solid-state drive, hard drive or other memory device. The patient data repository 107 can store patient-related data and treatment parameters, such as patient records, treatment session chronology, and disease documentation and photos. The system data repository 108 stores all system-related data and parameters, such as the system log, x-ray calibration data, and system diagnostics results. The patient data repository 107 and the system data repository 108 can be discrete devices or physically combined. One or more partitions can be used if the repositories 107 and 108 are combined, such as a single repository.

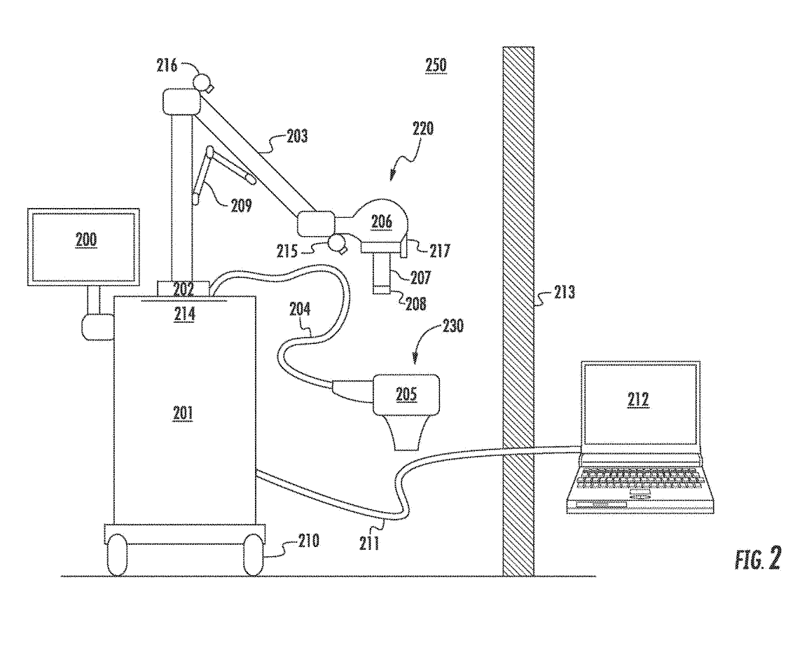

One embodiment of a VDW is shown in FIG. 2. The VDW system 250 can include a base unit 201 with various components mounted thereon or connected therewith. These components can include an imaging subsystem 230. In some scenarios, these components can also include a radiotherapy treatment device 220 for performing radiation therapy treatment in accordance with a received treatment plan.

The base unit 201 can be typically a compact unit such as one with a 30″×30″ footprint and can be mounted on casters 210 for ease of maneuverability. The base unit 201 can include a power lead for optionally providing power to all of the components housed in or connected to the base unit 201. In this regard, the base unit 201 can contain one or more computers for controlling the system 250 components and/or analyzing and processing data obtained from the system 250 components. A monitor 200 can also mounted to the base unit 201 for a user interface. Likewise, a terminal or an input device 214, such a as keyboard or mouse, can be included.

A mount 202 is provided on the base unit 201 for mounting the radiotherapy treatment device 220. The radiotherapy treatment device 220 can include a treatment arm 203 and treatment head 206, which can include removable or movable applicators 207, 208. The treatment arm 203 is articulated with appropriate retractable articulations 209. Although not shown in FIG. 2, additional articulations can also be provided at different points of system 250 to increase a number of degrees of freedom of placing and orienting treatment head 206.

A camera 216 can also be included to provide for remote operation or for documentation of treatment. A video-laser positioning system having camera 215 and laser or light pointer 217, which visibly marks a region with a crosshair that will receive radiotherapy treatment, can be provided. The camera 215 can capture low opacity images of the radiotherapy treatment head 206 and crosshairs of laser pointer 217 during treatment so that the exact positioning and orientation can be reproduced during subsequent treatments. In this regard, the video-laser positioning system can identify proper and precise positioning and orientation of treatment head 206. The video-laser positioning system can also allow for remote control and operation of the treatment arm 203 so that the treatment head 206 can be positioned precisely while the user is remote. In operation discussed below, the treatment arm 203 can be articulated and positioned to allow the treatment head to apply radiotherapy to a patient.

The imaging subsystem 230 can include at least one imaging head 205 attached via a corresponding lead 204 to the base unit 201 and data acquisition and processing machinery housed therein. The imaging head 205 can be a compact hand-held unit tethered to the base unit 201 by the corresponding lead 204. As such, the imaging head 205 can be freely moved to facilitate scanning different skin locations on the body of a patient. In operation, the imaging subsystem 230 can be used to collect both images and data of a diagnosis or treatment area before, during or throughout and after treatment. In some arrangements, an imaging head 205 can be mounted on the arm 203 instead of being provided separately.

Each imaging head can include components needed for supporting an imaging modality. For example, referring back to FIG. 1E, a first imaging head 205 can be provided that includes ultrasound component 103 and transducer 104 and a second imaging head 205 can be provided that includes optical imaging component 109 and image capture device 110. However, the present disclosure also contemplates combined functionality. That is, a single imaging head 205 can incorporate ultrasound component 103, transducer 104, optical imaging component 109, and image capture device 110.

Lead 211 can connect the system 250 to another computer 212 or use interface that can be positioned behind a shield 213 for remote operation of the system 250 or components of system 250, such as the radiotherapy treatment device 220.

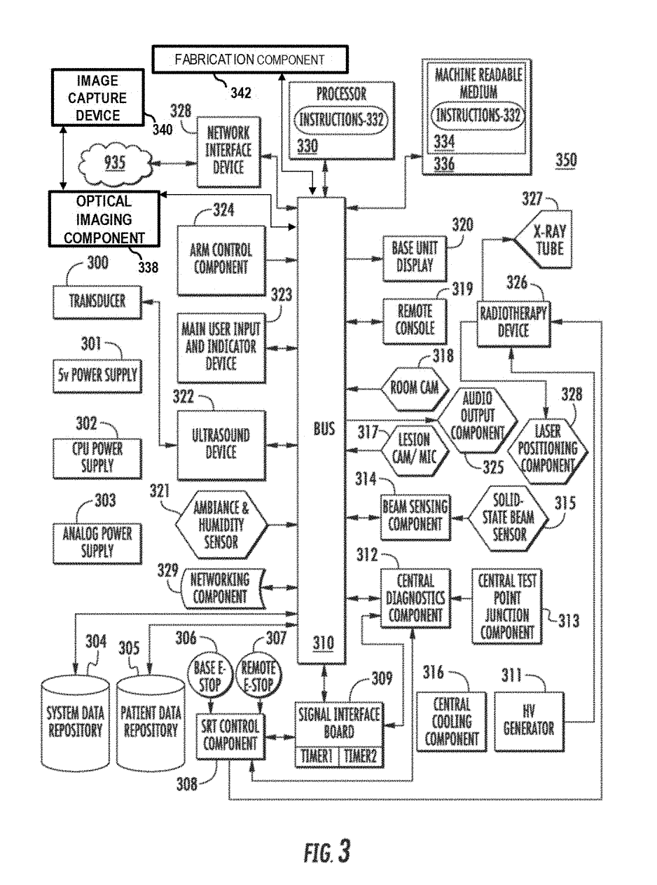

FIG. 3 illustrates a schematic view of various components and sub-components of a VDW system 350. The system 350 can include a bus 310 through which the various components can communicate with each other and/or the processor 330 (e.g., a central processing unit (CPU), a graphics processing unit (GPU), or both). The processor 330 can be connected to the bus 310 as shown in FIG. 3 or integrated therewith. Power supplies 301, 302, 303 can also be included.

The system 350 can be controlled and operated by processor 330 that runs the system 350 software or instructions 332, which controls the system 350 functions, verifies the safety mechanisms, and the service and calibration functions. The processor 330 can be in communication with a machine-readable medium 334, which can be static memory 336, on which is stored one or more sets of instructions 332 (e.g., software) embodying any one or more of the methodologies or functions described herein, including those methods illustrated herein. The instructions 332 may also reside, completely or at least partially, within the system data repository 304, static memory, or within the processor 330, or a combination thereof, during execution thereof by the system 350. The system data repository and patient data repository and the processor 330 also may constitute machine-readable media.

The processor 330 can be in communication with a motherboard having an appropriate amount of static or dynamic RAM, such as 4 GB of DRAM, in order to optimally support and accommodate the operating system, main software, and real-time system monitoring functions, together with efficient patient and data system handling and archiving. The system 350 software also communicates with the peripheral components, such as Ethernet, USB, and audio/video or via network interface card 338 in order to implement the system's user/machine interface and exchange data with external workstations and data repositories, such as electronic medical records (EMR), electronic health care records (EHR), hospital information systems (HIS), radiology information system (RIS), and picture archiving and communication systems (PACS), utilizing digital imaging and communications in medicine (DICOM) and health level 7 (HL7) communications and data structure protocols.

The system 350 can include storage mediums 304 and 305, such as solid state drives, hard drives or the like. Storage medium 304 can be the system data repository, which can include the operating system, the main system software, and system data and parameters archive. Storage medium 305 can be the patient data repository 305, which stores all patient-related data and records.

The system 350 can include a base unit that houses or otherwise provides various components of the system 350, including user interfaces. The base unit can include a base unit display device 320, such as an LCD display, and a base unit user input and indicator device 323, such as a terminal or a mouse. The system 350 can also include a remote console 319 that can be used to remotely control the system 350 so that a user does not need to be present during radiotherapy treatment. The base unit user input and indicator device 323 allows the user to interact with the system 350. The base unit user input and indicator device 323 can be utilized for initial patient data setup on the system 350 and for the ultrasound imaging of the patient's tumor at various stages of the disease before, during, and after the superficial radiotherapy period. Furthermore, the base unit user input and indicator device 323 can also be a terminal of the system 350 software. The diagnostics results and images, patient data, remote workstations topology, patient and room monitoring data, system service menus, system physics and calibration menus, and all system queues and alerts can be displayed on the base unit display device 320 or via the base unit user input and indicator device 323 as appropriate.

The system 350 can also include an ultrasound device 322 with a transducer 300. The ultrasound device can obtain structural or anatomic images of the treatment area or skin lesion of concern. With the ultrasound device 322 with a transducer 300, diagnostics of the area of concern can be processed. The ultrasound device 322 can be any ultrasound device capable of operating within an acceptable bandwidth. For example, the high frequency ultrasound device 322 can operate in a bandwidth of approximately 20 MHz to approximately 70 MHz, and may be implemented with an electro-mechanical, or solid state transducer. The system 350 can provide the ultrasound imaging device 322 at least partially integrated inside a system 350 housing coupled to bus 310 with a transducer head outside of the housing as shown in FIG. 2. The ultrasound device 322, and other components of the system 350, can be in communication with the bus 310 and the respective other components of the system 350 utilizing interface standards such as peripheral component interconnect (PCI/PCIe), universal serial bus (USB/USBII/USBIII), or Firewire. However, the present disclosure contemplates that any other interface and/or communications standards can be used.

The system 350 can also include an optical imaging component 338 with an optical image capture device 340. An optical imaging component 338 can obtain functional images of a three-dimensional volume comprising a treatment area or skin lesion of concern. With the optical imaging component 338 and image capture device 340, image data representative of the treatment volume of concern can be obtained and processed.

The optical imaging component 338 can include any type of image capture device now known or known in the future. According to one aspect, the optical imaging component can comprise an electronic image capture device 340 that captures incoming photonic radiation and converts same into electrical signals. An image capture device of this type can be comprised of a focusing element (e.g. a lens), a charge-coupled device (CCD) or a CMOS image sensor, and readout circuitry for acquiring the image data. Image capture devices as described herein are well known and therefore will not be described here in detail.

The system 350 can provide the optical imaging component 338 at least partially integrated inside a housing of system 250 coupled to bus 310 with an image capture device 340, outside of the housing as shown in FIG. 2. The optical image component 338 and other components of the system 350 can be in communication with the bus 310 and the respective other components of the system 350 utilizing interface standards such as peripheral component interconnect (PCI/PCIe), universal serial bus (USB/USBII/USBIII), or Firewire, to name a few. However, the present disclosure contemplates that any other interface and/or communications standards can be used.

The system 350 can further include a radiotherapy device 326 that includes an SRT X-ray tube 327. As discussed herein, the radiotherapy device 326 that includes an X-ray tube 327 can deliver radiation therapy to a particular region or area on a patient. The radiotherapy device 326 can be coupled with a high voltage generator 311 and a central cooling component 316.

The system 350 can also include a control component, such as superficial radiotherapy control component 308, for controlling the radiotherapy provided by radiotherapy device 326. The superficial radiotherapy control component 308 can control aspects of the radiation dosage, including timing, depth and intensity. In this regard, an arm control component 324 can also be provided with the system 350 and in communication with the superficial radiotherapy control component 308 and/or processor 330. The arm control component 324 can move, articulate or otherwise control positioning of the arm to which the radiotherapy device 326 and x-ray tube 327 are mounted. A base e-stop 306 and remote e-stop 307 can also be provided to provide local and remote emergency termination functions so that the radiotherapy device 326 can be stopped either locally or remotely.

Additionally, solid state beam sensing component 314 with a solid-state beam sensor 315 can be provided. In one embodiment, these components can be housed within the housing of X-ray tube 327. The solid state beam sensing component 314 with a solid-state beam sensor 315 provide the ability to obtain on demand and local analysis of the radiotherapy device 326 with X-ray tube 327. Utilizing the solid state beam sensing component 314 with a solid-state beam sensor 315, the radiotherapy device 326 with X-ray tube 327 can be tested to determine if the radiation output is consistent with the desired radiation output. In the event that there are discrepancies, the devices can be re-calibrated or otherwise serviced.

A central diagnostics component 312 can also be provided and can be interfaced with bus 310 and processor 330. The central diagnostic component 312 is also connected with a central test point junction conjunction 313 and additionally interfaces with a signal interface board 309 that is in turn connected to both the processor 330 through bus 310 and the superficial radiotherapy control component 308. The signal interface board 309 can also include a first and second timer for redundant time counting during the application of radiation therapy, which provides for added patient safety and accurate dosimetry calculation for the delivered therapy dose to the patient. In addition to the dual hardware timers, one or more additional software based timers can be utilized or invoked by system 350.

The central diagnostics module 312 is a systems diagnostic component that monitors the various system boards and components for failures and/or errors. The central diagnostics module 312 can generate alerts regarding the system status that can either be communicated with the user, or with the system installer or manufacturer for maintenance purposes.

Additional inputs can be connected to the processor 330 through bus 310 including a camera and/or microphone 317, an audio output component 325, such as a speaker, a room camera 318 for taking pictures or video of the patient, treatment areas and/or the treatment process. An ambiance and humidity sensor 321 can also be provided in the event that conditions may affect the treatment or any of the system 350 components. However, the arrangement is not limited in this regard. A fabrication component 342 for fabrication of a metal shield or template can also be connected to the bus 310 in some embodiments.

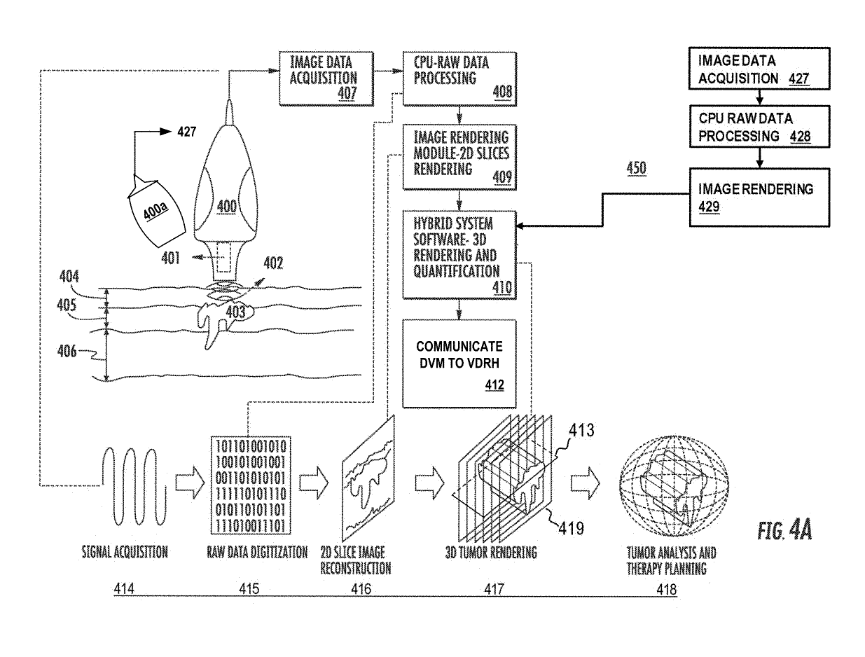

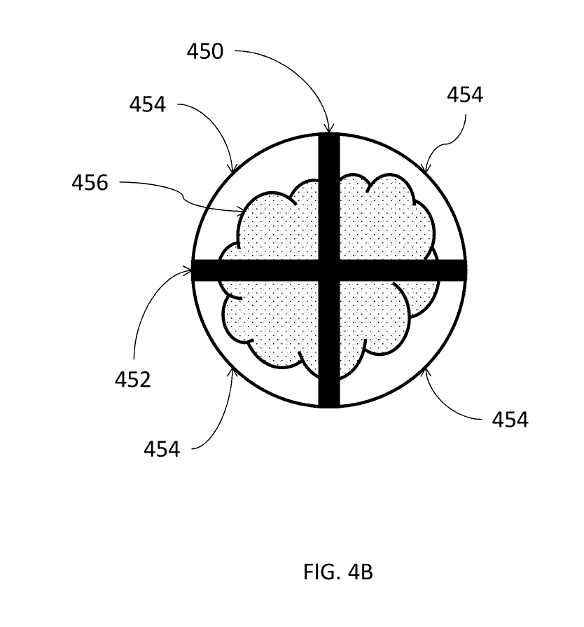

FIG. 4A illustrates an exemplary process 450 for generating a DVTM 29. The process involves use of with at least one imaging device 400 to facilitate the process. The at least one imaging device 4000 is used to provide images obtained using high frequency ultrasound and optical imaging. The method and process 450 can be used with VDW systems 150, 250 and 350, and their software that can be integrated with or operatively in communication with various system components as shown.

As shown in FIG. 4A, an ultrasound head or device 400 includes an ultrasound transducer 401 located in the head 400. High frequency ultrasound is used for the imaging, which provides a much clearer image in comparison to low frequency ultrasound, but which does not penetrate deeply into the skin. High frequency ultrasound can include frequency ranges of approximately 20 MHz to approximately 70 MHz, such as approximately 35 MHz to approximately 55 MHz.

In operation, the method and process 450 can begin with obtaining ultrasound image data. Using imaging device 400, an ultrasound beam 402 is aimed at an area of concern, such as lesion 403, through the epidermis 404, dermis 405 and subcutaneous fat 406. In use, the transducer 401 sends high frequency ultrasonic waves towards the epidermis 404, dermis 405, and subcutaneous tissue 406, where the tumor 403 is located in varying depth, circumference, and volume. The reflected ultrasonic waves 402 that hold the tumor's physical characteristic data (structural/anatomical data) are acquired by the transducer 401. For example, the structural/anatomical data collected can include a density characteristic of the tissue which is being imaged. A lesion will have a density that is different from surrounding healthy tissue and can therefore be easily differentiated within the captured image data. The difference in density can allow the location and boundaries of the lesion to be determined. As an example, a non-melanoma skin cancer lesion 403 is located as shown, and the lesion 403 may extend away from the epidermis 404 and into the subcutaneous fat 406.

Data and images 419 obtained by the ultrasound device 400 are acquired by an image data acquisition component at step 407 and pre-processed for further processing, as shown with the flow diagram of FIG. 4A. The image data acquisition 407 can be followed by processing at central processing unit (CPU) at step 408, in which the CPU processes the raw data captured by the image data acquisition at step 407. Thereafter, at step 409, the central processing unit can execute instructions of software to process the data to create and render 2-dimensional (2D) images, which are acquired in slices across the area imaged by the ultrasound device 400. As an aid to understanding, steps 407, 408 and 409 are graphically illustrated at 414, 415, and 416.

The data and images obtained by the ultrasound device comprise structural data that is useful for representing the skin cancer lesion 403. At 427-429, functional data is also obtained for the skin cancer lesion 403. The functional data can be acquired using an optical imaging component as described herein. Such optical imaging component can comprise a separate image capture head 400a or can be incorporated into the imaging device 400 so that both types of image data can be captured concurrently. Accordingly, the acquisition of functional data can begin at 427 with the acquisition of raw image data. The process can continue to 428 where the central processing unit can execute instructions in software to process the data so as to create or render a 2-dimensional image at 429.

Steps 427-429 can involve performing optical imaging using a spectroscopic imaging device to obtain a two-dimensional optical image 413. For example, the optical image can be obtained by using a technique such as multispectral imaging, hyperspectral imaging and/or Raman spectroscopy to capture and generate two-dimensional biological or functional image data for the tumor.

The process continues at step 410, which involves fusing the structural data obtained in steps 407-409 and comprising the 3D model, with functional data obtained in steps 427-429. This step is graphically shown in 417, which shows that the optical image 413 obtained in steps 427-429 can be a two-dimensional image which corresponds to an image plane that is essentially orthogonal to the two-dimensional image slices 419 comprising structural data obtained using high-frequency ultrasound. The image plane corresponding to the optical image 413 will generally correspond to the outer surface of the skin tissue. The nature of a particular skin lesion will be consistent throughout the lesion, so it is not necessary to capture the functional data associated with the cells at all skin depths.

According to one aspect, registration/location information associated with the optical imaging in step 427 and the image acquisition in step 407 can be utilized to fuse the optical image data with the 3D model. Thereafter, the model can be updated or enhanced to indicate both structural and functional information regarding the tumor. Methods for image registration are well known in the art and therefore will not be described in detail. However, it will be appreciated that the two images can be registered by using techniques involving fiducials or pattern recognition methods.

Once the images are registered, the information they contain can be combined into the fused 3D model comprising the DVTM 29. This can be done in a variety of ways. In some configurations, the structural image data can be provided in grayscale and the functional image data can be provided as a color overlay to indicate the functional information. In another configuration, the structural image data can be provided in grayscale or color and the functional information can be used to adjust the data in the structural image data. For example, the function information can be used to attenuate one or more color properties of data in the structural image data.

As noted above, the UHF imaging can differentiate tumor cells as having a density different from adjacent healthy/normal skin cells. Accordingly, in a subsequent data fusion process, the cells having a first density associated with a cancerous lesion can be highlighted, marked or otherwise displayed in a certain way to differentiate them from the adjacent healthy skin cells. For example, voxels associated with the cancerous skin cells can be displayed in a different color as compared to normal skin cells.

Once biological/functional nature of the tissue comprising the lesion has been determined using the optical imaging methodology, all of the cells which are identified as being associated with the lesion can be marked accordingly. Therefore, the image combining step can involve evaluating each voxel associated with the 3D model which was generated using ultrasound to determine whether a tissue density at particular voxel location differs from a density of healthy tissue at surrounding locations. If a particular voxel density at any skin depth corresponds to the lesion (e.g. cancerous tissue) then the voxel can be assigned a voxel color value associated with cancerous tissue. The voxel color can determine the color that the voxel is displayed as when rendered. But if a particular voxel density at any skin depth corresponds to healthy tissue, then it can be assigned a different voxel color value. Accordingly, the functional or biological information captured using a two-dimensional optical imaging technique can be extended or used to assign a color values to all of the voxels in the 3D model obtained by using ultrasound methodology. The result is a hybrid or fused 3D model containing information from both ultrasound and optical imaging methodologies.

A 3D construction and rendering engine can combine all or a portion of extracted 2D tumor slices obtained using ultrasound and optical imaging methods as described herein. The data from each scanning process can be merged and the system will fuse them into an integrated 3D model that represents and manifests the tumor's 3D anatomical and functional features. During the construction and rendering sequence, the 3D engine can apply geometrical corrections, smoothing and anatomical triangulation to the rendered 3D tumor model in order to achieve a correlation with the actual scanned or imaged tumor.

Thereafter, when the fused 3D model is rendered, the cancerous skin tissue can appear as a different color as compared to the surrounding healthy skin tissue. The particular color chosen for the cancerous tissue can be determined by the type of cancer. For example, tissue identified as a BCC can be assigned the color yellow, tissue identified as an SCC can be assigned blue, and tissue comprising a melanoma can be assigned the color red. Accordingly, when the fused 3D model is rendered to a treatment specialist, the boundaries and type of lesion can be easily apprehended.

In some configurations, the resulting fused 3D model may not provide a “real world” representation of the tumor. That is, the color and other properties of the fused 3D model may not correlate with what a physician or diagnostician is accustomed to reviewing in a microscope slide or other specimen during a biopsy. Accordingly, the present disclosure contemplates that in some configurations, to ease review and treatment planning, an additional translation or transformation may be used. That is, a transformation matrix can be provided for converting the raw fused 3D model into a 3D model that visually corresponds to what a physician or diagnostician is accustomed to reviewing in a microscope slide or other specimen during a biopsy. Once any final transformations are completed, the fused 3D tumor model comprising the DVTM 29 can be communicated at 412 to the VDRH 20 for tumor analysis and treatment planning 418 (e.g. treatment planning at an RTP workstation 47).

In some configurations it may not be desirable to acquire a large number of slices to generate the 3D tumor model. In particular, the 3D tumor model can be generated at the VDW from two 2D slices, as shown in FIG. 4B. That is, a first slice 450 and a second slice 452, perpendicular to each other, can be selected. Thereafter, the volume in the areas between the slices 454 can be extrapolated from the data associated with the first slice 450 and the second slice 452 and an estimated 3D model of the tumor 456 can be generated. In some configurations, the edges of the tumor in the first slice 450 and the second slice 452 can be identified and the estimated 3D model of the tumor 456 can be generated from only the data in the first slice 450 and the second slice 454 associated with the tumor in these slices.

The process illustrated in FIG. 4B expedites the 3D modeling process as only limited 2D data is required to generate the estimated 3D model of the tumor. In some scenarios, treatment planning does not require a highly accurate 3D model of the tumor, in which case the estimated 3D model of the tumor 456 will suffice. However, the present disclosure contemplates that in other configurations that a similar process can be performed using any number of 2D slices. In either scenario, the physical image data and functional image data will be fused as described herein with respect to FIG. 4A.

A DIPW 25, 30 as shown in FIG. 1C is comprised of a computer processing system which can include hardware and/or software, for tumor pathology analysis. A pathologist can use the DIPW to analyze the DVTM 29. This analysis can comprise study of the tumor structurally and functionally to identify the lesion type. Thereafter, the pathologist can generate a pathology report and use the facilities provided by the network and VDRH 20 to communicate the report back to the ISTF 21 (e.g. to the VDW or other computer system located at the ISTF 21).

In some scenarios, the DVTM 29 can also be used at an RTP workstation 45, 47 to perform radiotherapy planning. Accordingly, the VDRH 20 can also communicate a DVTM 29 and/or pathology report 31 to the RTP workstation as shown in FIG. 1D. In some scenarios, the DVTM 29 may include instructions as to a particular RTP site to which the DVTM is to be communicated. The radiotherapy planning 418 performed at the RTP workstation can include calculating the designated treatment area voxel, along with the pertinent dosimetry to be applied by a radiotherapy device. The dosimetry can include measurements and calculations of the absorbed dose in tissue resulting from the exposure to radiation. In one example, the appropriate treatment volume can be a spherical shape or a cylindrical shape. Alternatively, the appropriate treatment volume can be any other suitable shape that will leave appropriate treatment margins around the tumor. The accuracy provided by the hybrid imaging allows the treatment margins to be of the order of 10%, which is a significant improvement over the typical treatment margins used in Mohs surgery.

Appropriate therapy can be determined at the RTP workstation 45, 47 based on the DVTM and/or pathology report. An RTP workstation as described herein can include radiation therapy software to facilitate this process. Such software can include vector tables identifying the appropriate radiotherapy dosages for different sized tumors. This software can allow a therapy planner to precisely calculate therapy parameters, including treatment dosage based on the actual size of the tumor, including its depth and volume, rather than relying on the physician having to estimate the tumor size and depth based on experience and the visible surface area of the tumor.

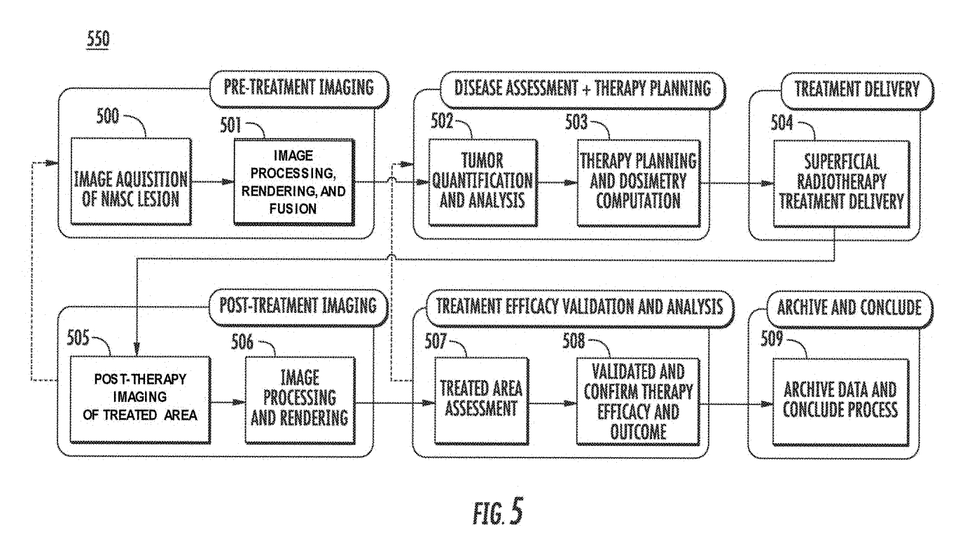

One embodiment of a method 550 of diagnosis, therapy planning, radiotherapy treatment, post treatment diagnosis and treatment validation and analysis is provided in a flow diagram of FIG. 5. Although the flow diagram illustrates the steps as sequential, the steps can be completed in different logical orders and some steps, or groups of steps, can be repeated as needed.

The method 550 can begin during a pre-treatment imaging stage at step 500 with image acquisition of an area of concern, such as a non-melanoma skin cancer (NMSC) lesion or a tumor. This step is utilized to acquire the structural and functional parameters of the lesion or tumor. Acquired data and images are then transferred to the image processing, rendering, and fusion step 501 to generate the DVTM 29. Steps 500 and 501 are performed at a VDW as described herein. As noted above in reference to FIG. 4A and the corresponding discussion, image data regarding structural features is subject to image processing and rendering and combined with image data regarding functional or molecular features at step 501 to generate a 3D model of the tumor and, optionally, of surrounding tissues.

The method then moves on to a disease assessment and therapy planning stage starting at step 502. The DVTM 29 is communicated to a VDRH which forwards the sample to a pathology site 32. At step 502, the lesion or tumor in the 3D model is assessed, defined, quantified, and diagnosed by a pathologist using a DIPW 25, 30. This can include any classifications for skin cancer type. The assessment at step 502 is based on both the structural and functional features of the 3D model. The 3D model provides a user, such as doctor or clinician, with a means to diagnose the skin cancer type instead of using the time consuming and invasive biopsy method. However, the present disclosure contemplates that automated diagnosis systems can be utilized, with or without a user confirmation step. Such automated diagnosis systems can utilize, for example, pattern recognition techniques to identifying one or more portions of the 3D model exhibiting signs of disease.

Based upon the initial imaging and analysis data, including any classification as to a type of skin cancer, the clinician and patient can decide to proceed with superficial radiotherapy to treat the diagnosed skin cancer lesion. Thus, during the disease assessment and therapy planning stage, tumor quantification and analysis can also occur at step 502 to identify a suitable treatment volume for the tumor that provides acceptable margins. Based on the 3D model and the treatment volume, a tumor volumetric model is provided. This portion of the process is advantageously performed at an RTP site 46 based on the DVTM 29, a pathology report 31, or both. Accordingly, the VDRH can communicate one or more of the foregoing to the RTP site where the information is analyzed to develop a radiotherapy treatment plan. This process can occur automatically if a pathology report indicates a lesion is cancerous, or can be requested by a physician at an ISTF based on a received pathology report.

With the tumor volumetric model, therapy planning and dosimetry computation can occur at step 503. Therapy planning and dosimetry computation is carried out at step 503 where the data and images passed from step 502 can be further manipulated and analyzed, including analysis of the tumor and therapy factors. The therapy planning can include not only the dosimetry computation, but also planning for the best location on the skin to reach the center of the tumor and the best angle of presentation for the treatment head. To plan treatment, the 3D tumor model can be orientated in correlation to the anatomy of the area to be treated. Physiological, topographic, and radiation therapy dosimetry parameters can be applied to compute and design the treatment plan, beam targeting and guidance, including the treated area voxel. A fractionation scheme and treatment head positioning on the patient can also be determined. The treatment plan can then completed. The completed treatment plan is communicated from the RTP site to the VDRH, from whence it is communicated back to the ISTF and the treating physician.

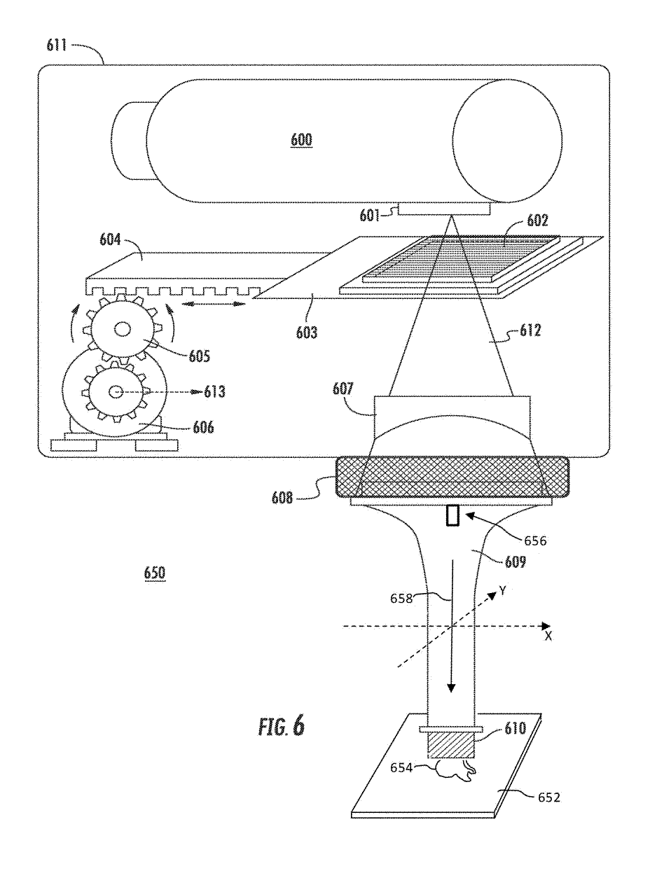

In some configurations, the treatment planning performed at the RTP site can involve generation of a mask or shield formed of a thin plate of material such lead. An exemplary mask or shield as described herein can be understood with reference to FIG. 6. As illustrated therein, an X-ray treatment head (e.g., treatment head 206) can comprise a housing 611 within which is disposed an X-ray tube 600. A Bremsstrahlung beam-hardening aluminum filter 607 can be disposed within the housing to control the X-ray beam 612 output from the x-ray tube 600. As shown, an X-ray beam aligned along a vector direction 658 can be applied by an applicator unit 609, which limits the overall cross-section of the beam. In order to further limit the amount of irradiated tissues, a mask 652 can be generated. The mask can be placed over the patient's body, where the mask includes an aperture or cutout portion 654. The cutout portion is sized and shaped to allow radiotherapy to be applied to a more limited area of the patient's body. The aperture or cutout area 654 is advantageously cut to the shape of the specific cancerous lesion but includes a further margin which extends a predetermined distance beyond the outer periphery of the cancerous cells. Only that portion of the X-ray beam that passes through the cutout will interact with the cancerous cells. Moreover, since the mask acts as a shield to radiation, high doses can be applied to the cancerous cells or lesion, since concerns with irradiating healthy tissues are significantly reduced.

According to one aspect, the aperture or cutout area 654 of the shield 652 can be designed based on the fused 3D hybrid image of the cancerous cells comprising the tumor. In other words, a therapy planner at an RTP workstation 45, 47 can use the DVTM 29 to determine an optimal shield pattern based on a selected beam vector and the hybrid image of the cancer, which has been generated using the techniques described herein. Thereafter, the RTP planner can use the RTP workstation to generate a scaled shield pattern as a data output (e.g., output using a network interface device 328) to facilitate fabrication of an optimal shield for use in treatment. The shield can then be manufactured manually or by suitable automated means.

For example, the data output data from the RTP system can be in a format suitable for a conventional Computer Numerical Control (CNC) type machine which uses a computer to control machine tools. Alternatively, the data format can be suitable for a 3D metal printer, capable of printing 3D metal parts. Consequently, the shield can be manufactured automatically and with high precision. In some configurations, where the treatment planning calls for radiotherapy to be provided at various angles, a different mask 652 can be provided for different angles or groups thereof, where the shape of the aperture 654 can be adjusted based on the 3D model and the radiotherapy angle to be used. Accordingly, this information can be included as part of a treatment plan.

The shield pattern generated by the RTP workstation can be communicated in the form of a data file to the ISTF where a fabrication component 111 can be used to fabricate the shield based on a data file. But in some scenarios it may be inconvenient for an ISTF (such as a dermatologist office) to fabricate the shield. This can be due to the hazardous nature of the materials that are used (e.g., lead plate), the lack of suitable equipment to generate a precisely fabricated shield, or simply a desire on the part of the treatment center to avoid becoming involved in metal fabrication work.

Accordingly, a technician at a mask fabrication site 48 can receive the shield information from the RTP workstation 47. The shield information can in some scenarios be communicated directly from the RTP site to the mask fabrication workstation 49 through network 24. However, the communication can also be coordinated through the Virtual Pathology Manager 26. In either scenario, the two dimensional pattern can be communicated to the mask fabrication workstation in a data file format which is suitable for controlling a mask fabrication machine 50. One example fabrication machine that can be used for this purpose can include a tabletop computer numerically controlled (CNC) router (e.g., a CNC machine). However, in other scenarios the fabrication machine can comprise a 3D printer. Thereafter, the fabricated shield or template can be fabricated using a mask fabrication machine, and delivered to the ISTF 21, so that it is available for use in treatment of a patient.

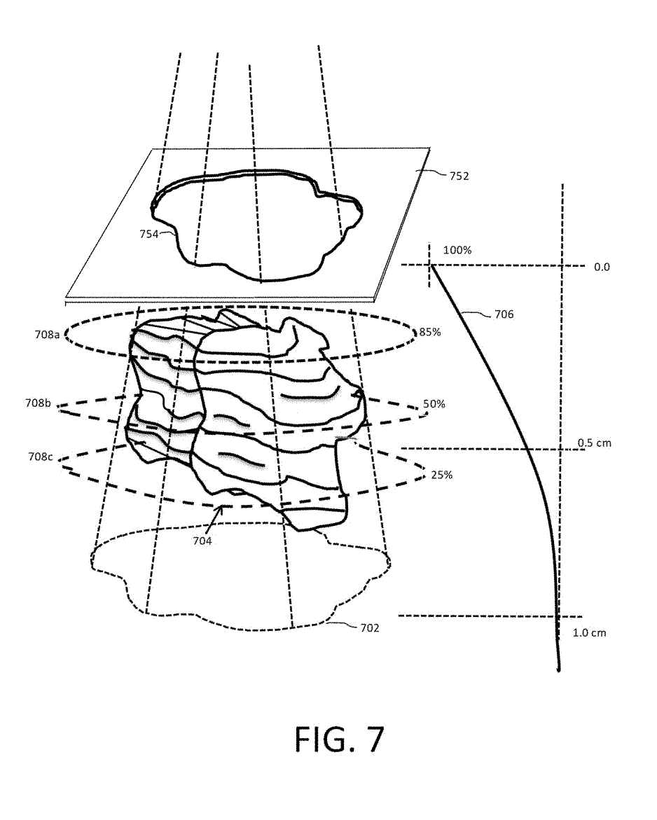

Referring now to FIG. 7, a three-dimensional pattern 702 of radiation can be synthesized or modeled by a processor in an RTP workstation 45, 47 whereby the three-dimensional pattern is an irregularly shaped volume is determined in accordance with the defined shield 752 and vector angle of applied radiation. Consequently, the three-dimensional pattern can visually show where radiation will be applied to underlying tissue as a result of the function of the shield. The irregularly shaped radiation pattern can be superimposed on a previously acquired two-dimensional or three-dimensional hybrid image of the cancerous cells 704 as described herein. The resulting composite image as shown in FIG. 7 is then presented to a treatment specialist on a display device (e.g., on a display device associated with the RTP workstation 45, 47 so it can be observed.

The treatment specialist can then use the displayed image to visualize a resulting three-dimensional pattern of radiation which will extend through the skin tissue when using the shield 652 and applicator unit 609. The shield can then be evaluated to determine whether the resulting three-dimensional beam representation is intersecting all of the desired cancerous cells at each skin depth. Accordingly, the treatment specialist can determine whether the cancerous cells are being properly dosed with radiation in accordance with a potential treatment plan.

From the foregoing it will be understood that the three-dimensional beam pattern 702 generated for visualization purposes will depend in part on a beam alignment or vector direction 658 of a radiation beam applied to the tissue and screened by the shield 652. A processor associated with the RTP workstation 45, 47 can specify information about the shield aperture 654 and the radiation applicator 609 (including position and beam vector direction). Thereafter such information can be used to model the beam pattern as shown in FIG. 7 and to show the extent of the three-dimensional volume of applied radiation (for visualization purposes). Such pattern can then be displayed in two-dimensions or three-dimensions. Advantageously, the displayed three-dimensional image comprising the modeled radiation beam 702 can be superimposed over the hybrid image of the cancerous tissues 704 so a treatment specialist can visually evaluate whether the beam will be applied to all portions of the tissue that are determined to be cancerous.

According to a further aspect, the visualization in FIG. 7 that is obtained by using the shield 652 and vector 658 data can be combined with a further graphical display of certain data. For example, such graphical display of data can be data that is useful for understanding a radiation dose which will be applied at various tissue depths relative to a surface of the skin. The graphical data can be unique to a particular radiotherapy device 326 and associated X-ray tube 327.

As is known, a particular X-ray tube (e.g., X-ray tube 600) used for delivery of radiation therapy to a patient will have a radiation output profile which is determined by various factors. These factors can include minor variations in the manufacture of the X-ray tube and in the radiation filters 607 that are used. Accordingly, the radiation characteristic of a particular radiotherapy delivery component will generally be unique to each particular radiation therapy device or machine. For example, a Percentage Depth Dose (“PDD”) plot 706 can be used to graphically show how much of a radiation dose from a particular radiation therapy machine will actually be delivered to skin tissue as a function of penetration depth. A PDD will vary in accordance with each radiotherapy system in accordance with minor variations in the X-ray tube and associated filters. Such a PDD characteristic is sometimes presented graphically. As will be appreciated, a radiation output profile such as PDD can be very important to a practitioner who needs to understand how applied radiation from the machine will interact with the tumor at various skin depths. In some embodiments, the PDD levels can be shown graphically as layers or rings 708a, 708b, 708c corresponding to the different radiation dose levels at different depths. The presentation of the PDD data in this way can allow a treatment specialist to more easily determine the amount of radiation which is to be applied to each portion of the cancerous cells 704.

Consequently, once a skin cancer has been imaged using the hybrid imaging methods described herein, it is advantageous to display the hybrid image (in two-dimensions or three-dimensions) to a treatment specialist together with a superimposed scaled graphical representation of the radiation dose profile (e.g. a PDD profile) 706 of a radiation therapy device which will actually be used to administer the radiation treatment. In this way, the treatment practitioner can visually evaluate the dose of radiation that will be delivered to various portions of the cancer as displayed, when using a particular radiation delivery device.

According to a further aspect a graphical representation of the radiation dose profile can be included in the three-dimensional representation of the radiation beam resulting from a particular shield pattern as described above. This graphic information can be combined with or overlaid on the hybrid image of the cancerous tissue as shown in FIG. 7 so that a proposed radiation treatment can be automatically evaluated by the RTP system. For example, a processor 330 associated with an RTP system 350 can evaluate whether all identified areas comprising cancerous skin cells will receive an adequate dose of radiation based on the foregoing. In some scenarios, the results of such evaluation can be communicated to a treatment specialist by highlighting or illuminating identified portions of the cancerous cells that will receive an inadequate dose of radiation. For example, such tissue areas can be highlighted in a different color to show the deficiency of the resulting three-dimensional beam pattern. All of the foregoing information can be used by the treatment specialist to develop a suitable treatment plan in accordance with step 503 in FIG. 5.

Upon completion of disease assessment 502 and therapy planning 503, the treatment plan can be communicated to a VDW system at the ISTF. The treatment plan can be communicated directly from RTP site to the ISTF. Alternatively, the treatment plan can be communicated to the VDRH 20 where it is matched to a particular ISTF that generated the DVTM and/or requested the treatment plan.

Once the treatment plan is received back at the ISTF, the process can continue at 504 where image-guided radiotherapy treatment fraction is actually delivered using a VDW 22 (e.g. a VDW system 150, 240, or 350). A radiotherapy component or device, such as component 220 from FIG. 2 or device 326 from FIG. 3, can be used to deliver treatment according to the treatment plan. Thus, the treatment plan can be read and interpreted and X-ray beam therapy can be delivered accordingly to the designated lesion. In this regard, the X-ray beam therapy is guided by the 3D imaging and dosimetry data from the treatment plan that was specifically created for the patient and the specific skin lesion to be treated.

In one arrangement, the components of the system 250 of FIG. 2 can be used to provide the image-guided radiotherapy treatment of step 504. The treatment head 206 and treatment applicators 307, 308 can be positioned over the patient's area to be treated utilizing the built-in video-laser positioning system 215, 217. The video-laser positioning system operates to align the treated area's video image with a low opacity snapshot of the previous treatment head 206 position together with crosshairs projected by laser 217 that are projected in both real-time and previous snapshot images. The system then ensures that the treated area's video image with laser crosshairs and the low opacity snapshot with the laser crosshairs are aligned together to the exact same position, which ensures an accurate and reproducible treatment head 206 positioning over the treated lesion. Once the treatment head 206 is in place, the user engages the system 250 to deliver the treatment fraction to the lesion. The timing, energy, and geometry of the beam are all guided by the image analysis, 3D tumor modeling, and the derived physics and dosimetry calculations and analysis.

For example, treatment can be provided in multiple, short fractionated treatment sessions. Each treatment fraction or session can be less than one minute long while delivering a dose of approximately 300 cGy to approximately 500 cGy per fraction. Depending on the prescribed total dose, the lesion can be treated with one or more fractions, such as around 12 to around 30 fractions. Additional or less fractions may be used.

A treatment series can include around 5 to around 30 fractions, per the protocol the physician prescribed for a particular lesion condition. Once the lesion is defined and identified, a treatment area is defined for the circumference. This circumference, in turn, can dictate the diameter of the applicator (such as applicators 207, 208 from FIG. 2 or applicator 609 from FIG. 6) that will be used. Generally, the selected applicator for treating a particular lesion circumference can be at least 20% larger in area and/or diameter of the lesion to be treated. The clinician can create a custom lead template or shield 652 that will be cut to the size and shape of the lesion with approximately a 15% extra margin, in order to ensure that the entire malignant area will be impacted by the x-ray beam. All healthy cells that will be exposed to the x-ray beam will generally successfully recover and regenerate, while the malignant cells will go through an apoptosis from which they will not recover.

As shown in FIG. 6, the template or shield 652 can be placed over the lesion, with the aperture or cutout 654 in the center of the treated area, and the selected applicator 609 will be latched onto the X-ray tube head. The treatment system can be set with the pertinent energy level and time span of the treatment (e.g., approximately 20 seconds to approximately 40 seconds), which are defined by vector tables, per the particular lesion's condition and fractionation scheme. The clinician can set the x-ray tube head vis-a-vis the applicator in position and aligned along a particular vector 658. Thereafter, the clinician can energize the system from the remote console, outside of the treatment room. The system can deliver the selected energy for the set time and can terminate treatment once the timers count to zero. The patient can be released and summoned for the next scheduled treatment session or fraction.

As will be discussed below, image-guided radiotherapy treatment step 504 can be repeated, or can be revised as needed. For instance, over the course of a treatment, a non-uniform shaped tumor may decrease in depth, width and overall size. Iterative treatments can be reduced in treatment size or intensity so that only the necessary amount of radiation is applied to as small a region as possible. In some scenarios, it can be advantageous to acquire further imaging data to characterize such changes in tumor depth, width and overall size. For example, the hybrid imaging methodology described herein can be used for this purpose. In some scenarios, additional or alternative imaging techniques can be used (such as LIDAR) to help to track the changes in the structure or anatomy of the tumor. The imaging data from these alternative methods can be combined or fused with imaging data acquired using other methods such as ultrasound and/or optical imaging methods as described. The resulting DVTS 29 can be communicated once again to the VDRH 20 for further analysis at an RTP site as described above.

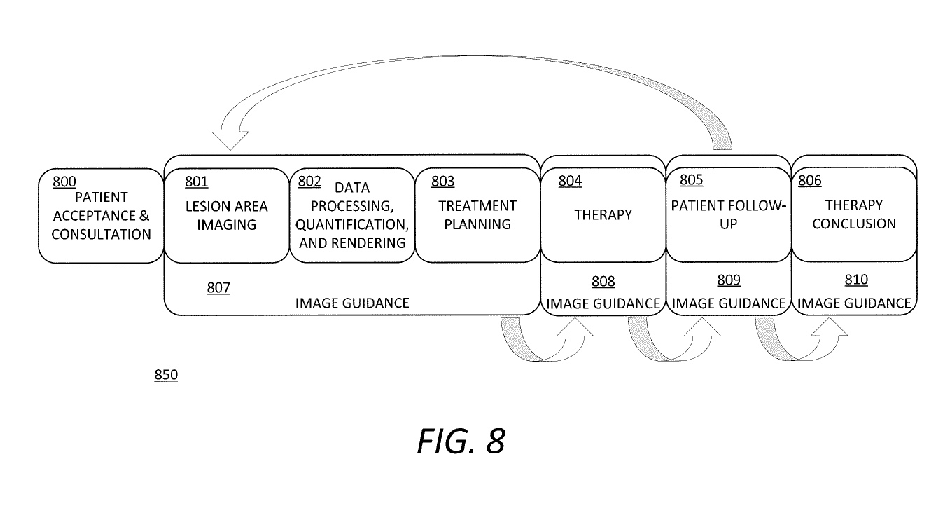

The method 550 can then move on to the post treatment imaging phase. The post treatment imaging phase can be an iterative lesion imaging procedure that is completed after the previous imaging and assessment of the lesion. Post treatment imaging provides the ability to track and evaluate the therapy progression and healing process of the treated lesion during the fractionated therapy process. The interval of the lesion imaging is determined by the clinician according to the protocol illustrated in FIG. 8 and discussed further below.

At step 505, the treated area can again be imaged, as during the pre-treatment imaging stage. Thus, the actions and operations of step 500 can be repeated to obtain post-treatment structural and functional data for the tumor. The post treatment images and data of the treated lesion can be processed and a new fused 3D model of the tumor can be rendered in step 506. During step 506, the actions and operations of step 501 can be repeated.

The method 550 can then move to the treatment efficacy validation and analysis of steps 507 and 508, which provide for an iterative analysis of the lesion's topography and volume over time throughout the span of treatment and upon treatment completion, during the follow-up sessions. At step 507, the post treatment images and data from steps 505 and 506 can be assessed to determine how the legion responds to therapy in comparison to the anticipated healing rate, according the prescribed dosimetry and fractionation scheme of the treatment plan. Step 507 can include one or more of the actions or operations of step 502 to fully analyze a tumor or lesion. In some scenarios, this can involve once again communicating an updated DVTM 29 to the RTP site 46.

As an example, if the newly acquired images and data indicates non-responsiveness, the treatment plan or other factors can be reviewed by a planner at the RTP site. In this regard, step 503 can be repeated where the treatment plan can be changed based on the post treatment imaging and assessment. On the other hand, if the newly acquired images and data indicates responsiveness, the treatment plan can be confirmed and treatment can progress. The steps provide an ongoing treated lesion assessment throughout the treatment and at its completion to evaluate the lesion evolution and its response to the therapy. The post treatment analysis can also be during the post-treatment follow-up sessions of the patient in order to document and validate or verify the full recovery and healing of the skin cancer lesion in the treated area.

Step 508, validation and confirmation of therapy efficacy and outcome can obtained. Step 508 can include image triangulation as a function of time and volume. By triangulating the 2D or 3D images, the size and shape of the tumor or lesion can be tracked. Also, volumetric analysis of the tumor over time can also be completed. The changes in the size, shape and volume of the tumor can be compared reviewed to determine effectiveness of the treatment, while factoring parameters of tumor transmutation and response to therapy. Thus, the clinician obtains an ongoing accurate assessment of the patient's response to therapy and can adjust the therapy if necessary in order to further optimize the patient's outcome. For instance, treatment session lengths or intensity can be decrease or increased. Step 508 can be completed locally by the clinician in a non-invasive manner, without any discomfort to the patient or the necessity for ancillary lab and pathology services.

At step 509, patient records can be stored and the method 550 completed. The records can include patient record data and images, results, and summary reports that illustrate the patient's disease state from procedure commencement to its ultimate conclusion. The records can be stored to local and networked record storage repositories.

Any of the steps can be repeated as needed. For instance, a tumor may require multiple treatment sessions before treatment is completed. The imaging steps, assessment and planning steps and the treatment steps may be repeated multiple times.

As all of the DVTM generating steps and treatment steps can be carried out with a single machine in a physician's office, this greatly cuts down the time, inconvenience and expense associate with diagnosis and treatment of non-melanoma skin cancer. Additionally, as it reduces or even removes the need for Mohs surgery, the patient's subsequent healing time and scarring is much reduced.

In certain configuration, the steps can be implemented via a workstation associated with the radiotherapy system. For example, computer 212, as shown in FIG. 2, can be configured with a computer program to guide the user, such as a doctor, through the various steps of FIG. 5. Thus, from computer 212, the user can perform tumor imaging and modeling processes, communicate a DVTM to a VDRH, receive pathology reports, treatment plans and mask data, and carry out treatment plans. In other configurations, the workstation can be deployed on a remote system. For example, on another computer connected over a network to the radiotherapy system.

Turning once again to FIG. 6, a solid state beam sensor and beam sensing component 650, such as solid state beam sensor 313 and beam sensing component 312 of FIG. 3, or solid-state X-ray beam sensing component 102 of FIG. 1E, can be provided. These components can be incorporated in an X-ray tube housing 611, such as the X-ray tube in treatment head 206 of FIG. 2.

The component or device 650 can include retractable support structure 603, 604 that can also be incorporated in X-ray tube housing 611. Also provided can be a x-ray imaging array detector, such as solid-state x-ray detector array 602, which is located between the X-Ray tube 600 with x-ray tube alignment 601, and a Bremsstrahlung beam-hardening aluminum filter 607. The solid-state detector array 602 that is mounted on the retractable support structure 603 can be utilized to sense the x-ray beam 612 output from the x-ray tube 600.

The retractable support structure 603 can move the solid-state detector array 602 between an X-ray testing position, as shown in FIG. 4A, and a non-testing position. In the non-testing position, the solid-state detector array 602 and/or the retractable support structure 603 is retracted or moved away from the field of emitted x-rays such that they do not absorb, block or otherwise interfere with radiation beams that are emitted from x-ray tube 600.

When the x-ray beam 612 is detected, the solid-state detector array 602 can sense characteristics of the radiation emitted from the x-ray tube 600. The detector array 602 can be used to generate a matrix-like image of the circumference of X-ray beam 612, together with the intensity of the x-ray beam's particles.

The x-ray imaging array detector can be a one-line array or a matrix array of solid state x-ray detectors that acquire and gather characteristics of the beam during the a check or testing procedure. One characteristic is the beam shape integrity, which validates that the x-ray tube output is indeed homogenous and without flaws. Another characteristic is the beam intensity that can be measured by centigray (cGy), or kilovoltage (kV) units. Other characteristics sensed by the x-ray imaging array detector include the cross section or shape of the beam. The x-ray imaging array detector can also accurately measure the photons emitted from the x-ray tube 600. Further, the x-ray imaging array detector can also be used to determine whether the x-ray tube port 601 is properly aligned or if realignment is needed.

The collected data can be communicated to the beam sensing component, such as beam sensing component 312 of FIG. 3 that pre-processes the data. The pre-processed data can be communicated with a processor, such as processor 330, for further analysis and visualization. The solid state beam sensor and beam sensing component can be utilized as a daily quality control tool and for overall system diagnosis purposes. For instance, the solid state beam sensor and beam sensing component may detect a difference between the programmed radiation and what is output from the X-ray tube 600. Detection can allow for maintenance to ensure desired treatment dosages are delivered. Still further, testing can be automated before use such that the emitted x-rays and/or alignment of the x-ray tube port 601 is confirmed prior to each use.

The retractable support structure 603, 604 can include at least one motor or actuator 613 and positioning components 605, 606. The motor or actuator 613 can be controlled via a processor, such as processor 330 with the beam sensing component 314. The motor or actuator 613 can move the solid-state detector array 602 between the X-ray testing position and the non-testing position.

FIG. 6 also shows a removable treatment head or applicator 609 with tip 610 that can be changed to suit the treatment area and depth needed. Suitable treatment heads that may be used in the component 650 can include treatment applicators, ranging from approximately 1 cm through approximately 7.3 cm diameter heads at approximately 15 cm SSD, and also approximately 1 cm through approximately 12.7 cm diameter heads at approximately 25 cm SSD. The removable head 609 can be used with radiotherapy devices 220 and 326.

FIG. 8 provides a flow chart of patient and treatment management protocol 850. When a patient first arrives at a doctor or clinician, the patient acceptance and consultation step 800 can occur. In this step, the patient is registered in the practice's workflow management system that can be integrated with a hybrid image-guided superficial radiotherapy system that can employ protocols such as DICOM and HL7, such as the system 350 illustrated at FIG. 3 or any suitable system.

With registration, the patient can be scheduled for the initial prognosis and consultation with the clinician. During initial prognosis and consultation, the patient can be scanned by an imaging device at 801 to obtain structural and functional information, as part of the image guidance phase 807. The scanning provides the clinician and the patient an assessment of any lesions and the disease state before making a decision on the recommended and preferred treatment path. The scanned lesion image data is then processed and reconstructed at step 802 to render the 2D image slices of the scanned area that contain the 2D cross-sections of the tumor. If desired, these images can be reviewed. The 2D tumor data can also converted to a 3D volumetric model and rendered to derive all the volumetric and physiological data of the tumor out of the scanned lesion, all for use in the next step.

Treatment planning step 803, involves communicating the DVTM to an RTP site which then uses the quantified data to calculate and generate a personalized treatment plan for the patient and the treated lesion.

Therapy can then commence at step 804, while imaging is still being applied 808 in varying intervals according to the clinician's and treatment plan's protocol to monitor and guide the course of the therapy throughout the prescribed fractions and entire treatment span.

Patient follow-up sessions 805 can be conducted to verify and monitor the full recovery of the treated lesion. As an example, follow-up sessions can be completed months or years after the last therapy session to monitor the area of a treated lesion. This stage is also being image-guided 809 with images obtained with a high frequency ultrasound device in order to add further validity to the treatment's outcome and to verify that indeed the lesion is completely cured and gone.

When the entire therapy sequence is complete at step 806, the patient data and all pertinent image-guidance data 810 is archived and submitted to the medical record management systems and the healthcare management systems. By generally the entire treatment process including non-invasive imaging as a substitute to invasive, time consuming, and expensive biopsies, the patient management protocol is being dramatically enhanced and improved, which offers benefits to all entities and parties involved, including the patient, the clinician, and the healthcare system as a whole.

The systems, methods and devices include broader applications beyond treating lesions or skin cancer. For instance, the systems, methods and devices can be utilized as intra operative radiotherapy in surgical environments to treat other cancers or lesions when their respective tumors are surgically removed. In addition to removal of a tumor, an excised area can imaged, analyzed and treated with the systems, methods and devices herein, such as treating an excised area with one or more 21 Gy fractions before the patient is sutured.

In some arrangements, the systems, devices, methods and protocols can be employed for relatively superficial tumors that are not skin cancers, such as certain breast cancers, in which case the treatment head can include a surgical catheter for insertion beneath the skin, together with a small scale spherical treatment head.

It is important to note that the methods described above may incorporate any of the functionality, devices, and/or features of the systems described above, or otherwise, and are not intended to be limited to the description or examples provided herein.

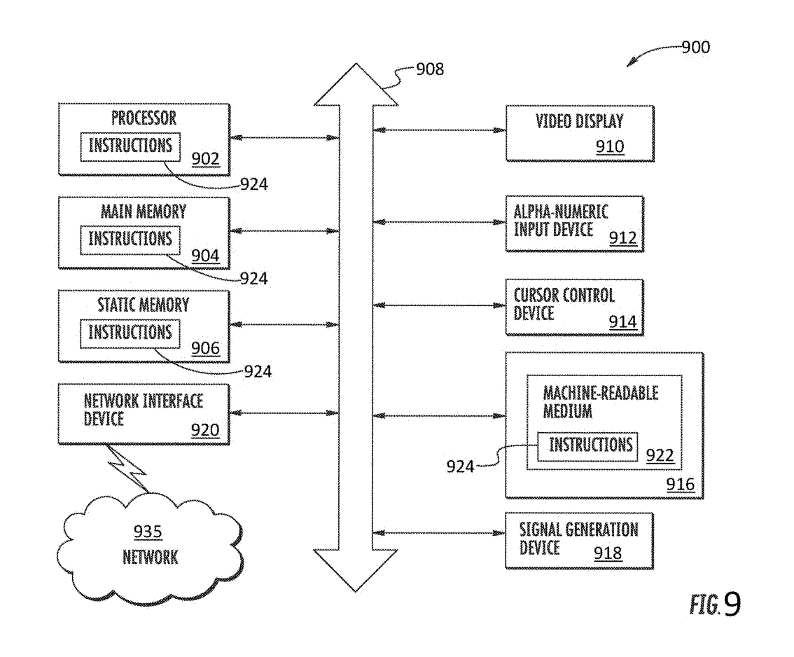

Referring now also to FIG. 9, at least a portion of the methodologies and techniques described with respect to the exemplary embodiments can incorporate a machine, such as, but not limited to, computer system 900, or other computing device within which a set of instructions, when executed, may cause the machine to perform any one or more of the methodologies or functions discussed above. The machine may be configured to facilitate various operations conducted by the systems illustrated at 1A-1E and FIGS. 2-4. For example the computer system 900 can be used to implement one or more of a VDW, a DIPW 25, 30, an RTP workstation 45, 47, and a mask fabrication workstation 49.

In some embodiments, the machine operates as a standalone device. In some embodiments, the machine may be connected using network 935 to other machines. The machine may operate in the capacity of a server or a client user machine in server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 900 may include a processor 902 (e.g., a central processing unit (CPU), a graphics processing unit (GPU, or both), a main memory 904 and a static memory 906, which communicate with each other via a bus 908. The computer system 900 may further include a video display unit 910 (e.g., a liquid crystal display (LCD), a flat panel, a solid state display, or a cathode ray tube (CRT)). The computer system 900 may include an input device 912 (e.g., a keyboard), a cursor control device 914 (e.g., a mouse), a disk drive unit 916, a signal generation device 918 (e.g., a speaker or remote control) and a network interface device 920.

The disk drive unit 916 may include a machine-readable medium 922 on which is stored one or more sets of instructions 924 (e.g., software) embodying any one or more of the methodologies or functions described herein, including those methods illustrated above. The instructions 924 may also reside, completely or at least partially, within the main memory 904, the static memory 906, or within the processor 902, or a combination thereof, during execution thereof by the computer system 900. The main memory 904 and the processor 902 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine readable medium 922 containing instructions 924 so that a device connected to the communications network 935 can send or receive voice, video or data, and to communicate over the network 935 using the instructions. The instructions 924 may further be transmitted or received over the network 935 via the network interface device 920.

While the machine-readable medium 922 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present disclosure.

The term “machine-readable medium” shall accordingly be taken to include, but not be limited to: solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. In one embodiment, the machine readable storage medium may be a machine readable storage device or a computer readable device. Accordingly, the disclosure is considered to include any one or more of a machine-readable medium or a distribution medium, as listed herein and including art-recognized equivalents and successor media, in which the software implementations herein are stored.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments. Rather, the scope of the invention should be defined in accordance with the following claims and their equivalents.

What is claimed is:

1. A virtual dermatology system, comprising:
a Virtual Dermatopathology Regional Hub (VDRH) comprised of a computer processor which implements a Virtual Pathology Manager (VPM);
the VDRH configured to communicate over at least one data network to
receive at the VPM a plurality of Detailed Virtual Tissue Models (DVTMs) of imaged skin lesions from one or more Virtual Dermatology Workstations (VDWs);
selectively communicate from the VPM each of the plurality of DVTMs to one or more pathology sites, each comprising one or more Dermatopathology Information Processing Workstations (DIPWs) which are configured to display the DVTM for analysis by a pathologist;
receive at the VPM from one or more of the pathology sites a plurality of pathology reports generated using said DIPWs and respectively corresponding to the plurality of DVTMs; and
selectively communicate from the VPM the pathology reports to the imaging facilities which generated respectively the DVTM upon which each said pathology report is based; and
wherein said one or more VDWs are each comprised of a hybrid imaging system which includes
a structural imaging component adapted to image structural features of the skin lesion;
an optical imaging component adapted image functional features of the skin lesion; and
an image fusion component which is adapted to combine the images obtained by the structural imaging component and the optical imaging component to form a 3D virtual model comprising the DVTM.

2. The virtual dermatology system according to claim 1, wherein the VPM performs managing and scheduling operations so that the plurality of DVTMs are distributed among the plurality of pathology sites for efficient processing.

3. The virtual dermatology system according to claim 2, wherein the DVM is configured to track at least one pathology site selection criteria selected from the group consisting of (1) the number of DVTMs which have been sent to each said pathology site for study, (2) the date and time each of the DVTMs were sent to each said pathology site, (3) a backlog indicating the number of DVTMs sent to each said particular pathology site for which pathology reports have not yet been generated, (4) a number of DIPW available at each said pathology site, (5) a number of pathologists available at each said pathology site, and (6) the expertise of the pathologists who are present at each said pathology site.

4. The virtual dermatology system according to claim 1, wherein the VPM is further configured to selectively utilize the data network to communicate at least one of the DVTM and a corresponding one of the pathology reports to at least one Radiation Therapy Planning (RTP) site, each RTP site comprising one or more RTP workstations adapted for radiation therapy planning.

5. The virtual dermatology system according to claim 4 wherein the VPM performs managing and scheduling operations so that the one or more of the pathology reports and the DVTM corresponding to each said pathology report are distributed among the plurality of RTP sites for efficient processing.

6. The virtual dermatology system according to claim 4, wherein the VPM is further configured to receive at the VPM from one or more of the RTP sites a plurality of radiation therapy plans generated using said RTP workstations.

7. The virtual dermatology system according to claim 6, wherein the VPM is further configured to selectively communicate the radiation therapy plan from the VPM to the imaging facility which generated the DVTM upon which the radiation therapy plan has been determined.

8. The virtual dermatology system according to claim 4, wherein said RTP workstation is adapted to display a three-dimensional radiation pattern superimposed over said DVTM for the radiation therapy planning.

9. The virtual dermatology system according to claim 8, wherein RTP workstation is adapted to selectively modify the three-dimensional radiation pattern in accordance with a shield model to visually show on a display where radiation will be applied to skin tissue when the shield is present.

10. The virtual dermatology system according to claim 9, wherein the RTP workstation is adapted to generate at least one data file output representative of a selected shield model, from which an actual shield can be automatically manufactured.

11. The virtual dermatology system according to claim 9, wherein, wherein the VPM is further configured to receive at the VPM from one or more of the RTP sites the data file output representative of a selected shield model, and selectively communicates same to at least one of the imaging facilities.

12. The virtual dermatology system according to claim 1, wherein the VPM performs an automated preliminary analysis of the DVTM to determine the most appropriate pathology site for receiving the DVTM for analysis.

13. A method for virtual dermatology, comprising:
using a data network to receive at a Virtual Dermatopathology Regional Hub (VDRH) one or more Detailed Virtual Tissue Models (DVTMs) generated at one or more Virtual Dermatology Workstations (VDWs);
implementing by means of at least one computer processor at the VDRH a Virtual Pathology Manager (VPM) which
receives the one or more DVTMs;
selectively communicates each of the DVTMs to at least one pathology site comprising one or more Dermatopathology Information Processing Workstations (DIPWs) which are configured to display the DVTM for analysis by a pathologist;
receives from the one or more of the pathology sites a plurality of pathology reports generated using said DIPWs and respectively corresponding to the plurality of DVTMs; and
selectively communicates the pathology reports to the imaging facilities which generated respectively the DVTM upon which each said pathology report is based;
wherein the DVTM is a 3D virtual model comprised of a fused image based on structural imaging adapted to image structural features of the skin lesion and an optical imaging component adapted image functional features of the skin lesion.

14. The method according to claim 13, further comprising performing with the VPM managing and scheduling operations so that the plurality of DVTMs are distributed among the plurality of pathology sites for efficient processing.

15. The method according to claim 13, further comprising selectively using the VPM to communicate from the VPM at least one of the DVTM and a corresponding one of the pathology reports to at least one Radiation Therapy Planning (RTP) site, each RTP site comprising one or more RTP workstations adapted for radiation therapy planning.

16. The method according to claim 15, further comprising performing managing and scheduling operations with the VPM so that the one or more of the pathology reports and the DVTM corresponding to each said pathology report are distributed among the plurality of RTP sites for efficient processing.

17. The method according to claim 15, further comprising receiving at the VPM from one or more of the RTP sites a plurality of radiation therapy plans generated using said RTP workstations.

18. The method according to claim 17, further comprising selectively communicating the radiation therapy to the imaging facility which generated the DVTM upon which the radiation therapy plan has been determined.

19. The method according to claim 16, wherein said RTP workstation displays a three-dimensional radiation pattern superimposed over said DVTM for the radiation therapy planning.

20. The method according to claim 19, wherein RTP workstation is adapted to selectively modify the three-dimensional radiation pattern in accordance with a shield model to visually show on a display where radiation will be applied to skin tissue when the shield is present.

21. The method according to claim 20, wherein the RTP workstation is adapted to generate at least one data file output representative of a selected shield model, from which an actual shield can be automatically manufactured.

22. The method according to claim 20, further comprising receiving at the VPM from one or more of the RTP sites the data file output representative of a selected shield model, and selectively communicating the selected shield model to at least one of the imaging facilities.

23. The method according to claim 13, further comprising performing an automated preliminary analysis of the DVTM at the VPM to determine a pathology site to which the DVTM should be communicated for analysis.
